(19) United States
(12) Patent Application Publication
(10) Pub. No.: US 2024/0398362 A1
(43) Pub. Date: Dec. 5, 2024

(54) ULTRA-WIDE 2D SCOUT IMAGES FOR FIELD OF VIEW PREVIEW

(71) Applicant: Medtronic Navigation, Inc., Louisville, CO (US)

(72) Inventors: Amir Eftekhar, Sudbury, MA (US); Erica C. Freund, Cambridge, MA (US); Elizabeth Rose Rooney, Waltham, MA (US)

(21) Appl. No.: 18/626,159

(22) Filed: Apr. 3, 2024

Related U.S. Application Data
(60) Provisional application No. 63/469,769, filed on May 30, 2023.

(51) Int. Cl. A61B 6/42; A61B 6/00

(52) U.S. Cl. CPC A61B 6/4208; A61B 6/488

(57) ABSTRACT

A system may perform a scan process of a patient anatomy according to an extended width mode of capture. The system may generate a fused localization image of the patient anatomy based on performing the scan process. The system may capture an image volume including at least a portion of the patient anatomy based on one or more target boundaries associated with the fused localization image.

### CROSS-REFERENCE TO RELATED APPLICATIONS

The present application claims the benefit of and priority to U.S. Provisional Application No. 63/469,769 filed on May 30, 2023, entitled “ULTRA-WIDE 2D SCOUT IMAGES FOR FIELD OF VIEW PREVIEW”, which application is incorporated herein by reference in its entirety.

### FIELD OF INVENTION

The present disclosure is generally directed to imaging, and relates more particularly to surgical imaging.

### BACKGROUND

Surgical robots may assist a surgeon or other medical provider in carrying out a surgical procedure, or may complete one or more surgical procedures autonomously. Imaging may be used by a medical provider for diagnostic and/or therapeutic purposes. Patient anatomy can change over time, particularly following placement of a medical implant in the patient anatomy.

### BRIEF SUMMARY

Example aspects of the present disclosure include:

An imaging system including: a processor coupled with the imaging system; and memory coupled with the processor and storing data thereon that, when executed by the processor, enable the processor to: perform a scan process of a patient anatomy according to an extended width mode of capture; generate a fused localization image of the patient anatomy based on performing the scan process; and capture an image volume including at least a portion of the patient anatomy based on one or more target boundaries associated with the fused localization image.

Any of the aspects herein, wherein the data executable to perform the scan process according to the extended width mode of capture is further executable to: capture a set of localization images associated with the patient anatomy in association with the extended width mode of capture; and merge the set of localization images, wherein: the fused localization image is generated based on merging the set of localization images; and the portion of the patient anatomy is included in one or more localization images of the set of localization images.

Any of the aspects herein, wherein the data executable to capture the image volume is further executable to compensate for parallax associated with: a point of view associated with capturing a first localization image of the set of localization images; and a second point of view associated with capturing a second localization image of the set of localization images.

Any of the aspects herein, wherein the data is further executable by the processor to establish one or more boundaries associated with the fused localization image based on: a first boundary associated with a first localization image of the set of localization images; and a second boundary associated with a second localization image of the set of localization images.

Any of the aspects herein, wherein the data is further executable by the processor to: generate movement data associated with positioning or orienting at least one of a radiation source, a detector, and a rotor of the imaging system in association with capturing the image volume, wherein generating the movement data is based on the one or more target boundaries, target coordinates associated with the fused localization image, spatial coordinates associated with the image volume, or a combination thereof.

Any of the aspects herein, wherein the data is further executable by the processor to: display guidance information associated with capturing the image volume at a user interface associated with the imaging system, wherein the guidance information includes at least one of the movement data and the spatial coordinates.

Any of the aspects herein, wherein the data is further executable by the processor to: control movement of at least one of the source, the detector, and the rotor based on the movement data.

Any of the aspects herein, wherein generating the movement data is based on: the one or more target boundaries associated with the fused localization image; orientation information associated with the fused localization image; and a system geometry associated with the imaging system.

Any of the aspects herein, wherein the data is further executable by the processor to generate one or more settings associated with capturing the image volume based on: the one or more target boundaries associated with the fused localization image; target coordinates associated with the fused localization image; and image data associated with the fused localization image.

Any of the aspects herein, wherein: a subject including the patient anatomy is positioned along an isocenter of the imaging system; and a virtual isocenter associated with capturing the image volume is different from the isocenter of the imaging system.

Any of the aspects herein, wherein the data is further executable by the processor to: generate a multiple field of view representation of the patient anatomy based on the one or more target boundaries associated with the fused localization image, image data included in the fused localization image, and the image volume, wherein the multiple field of view representation includes a preview representation of the image volume.

Any of the aspects herein, wherein capturing the image volume is in response to at least one of: a user input associated with the fused localization image; and a second user input associated with a multiple field of view representation of the patient anatomy, wherein the multiple field of view representation includes a preview representation of the image volume.

Any of the aspects herein, wherein the fused localization image includes a two-dimensional image.

Any of the aspects herein, wherein the image volume is based on three-dimensional system coordinates of the imaging system.

Any of the aspects herein, wherein a width of the fused localization image is equal to or less than 80 centimeters.

A system including: an imaging device; a processor; and memory coupled with the processor and storing data thereon that, when executed by the processor, enable the processor to: perform a scan process of a patient anatomy according to an extended width mode of capture; generate a fused localization image of the patient anatomy based on performing the scan process; and capture an image volume including at least a portion of the patient anatomy based on one or more target boundaries associated with the fused localization image.

Any of the aspects herein, wherein the data executable to perform the scan process according to the extended width mode of capture is further executable to: capture a set of localization images associated with the patient anatomy in association with the extended width mode of capture; and merge the set of localization images, wherein: the fused localization image is generated based on merging the set of localization images; and the portion of the patient anatomy is included in one or more localization images of the set of localization images.

Any of the aspects herein, wherein the data is further executable by the processor to: generate movement data associated with positioning or orienting the imaging device in association with capturing the image volume, wherein generating the movement data is based on the one or more target boundaries, target coordinates associated with the fused localization image, spatial coordinates associated with the image volume, or a combination thereof.

A method including: performing, at an imaging system, a scan process of a patient anatomy according to an extended width mode of capture; generating, at the imaging system, a fused localization image of the patient anatomy based on performing the scan process; and capturing, at the imaging system, an image volume including at least a portion of the patient anatomy based on one or more target boundaries associated with the fused localization image.

Any of the aspects herein, wherein performing the scan process according to the extended width mode of capture includes: capturing a set of localization images associated with the patient anatomy in association with the extended width mode of capture; and merging the set of localization images, wherein: the fused localization image is based on merging the set of localization images; and the portion of the patient anatomy is included in one or more localization images of the set of localization images.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIGS. 1A through 1D illustrate examples of a system in accordance with aspects of the present disclosure.

FIGS. 2A through 2G illustrate examples of generating an extended localization image and capturing an image volume in accordance with aspects of the present disclosure.

FIGS. 3A through 3E illustrate examples of generating an extended localization image and capturing an image volume in accordance with aspects of the present disclosure.

FIGS. 4A and 4B illustrate examples of extended localization images in accordance with aspects of the present disclosure.

FIG. 5 illustrates an example of a process flow in accordance with aspects of the present disclosure.

FIG. 6 illustrates an example of a process flow in accordance with aspects of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or 19 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Some imaging systems may support capturing “scout images” (also referred to herein as “localization images”) prior to a subsequent scan. For example, scout images may provide a user with a survey of a region of interest. The scout images may provide anatomical information based on which the system or a user may localize a target patient anatomy.

Some imaging systems may support a long scan process capable of generating a multidimensional “long scan” (also referred to herein as a “long film,” “long film image,” or “pseudo-panoramic image”) of a patient anatomy using an imaging device. For example, performing a long scan may produce a long film based on multiple images captured by an imaging device, which may provide a relatively longer or wider image compared to an individual image captured by the imaging device.

Some imaging systems may support capturing an image volume of a patient anatomy using an imaging device. In some radiological imaging workflows associated with taking a 3D image volume of a patient anatomy, the workflow includes acquiring capturing 2D “scout images” (also referred to herein as “localization images”) prior to a subsequent scan for capturing the 3D image volume. For example, based on the 2D scout images, a user (e.g., radiology technician) may confirm that the anatomy has been captured for a surgical procedure. Some workflows include acquiring a 2D anterior-posterior image and a 2D lateral image to ensure the patient is centered within a target volume to be captured in the 3D image volume.

The O-Arm® imaging system provided by Medtronic Navigation, Inc. supports providing a field of view (FOV) preview feature (also referred to herein as a FOV preview representation and a multiple FOV representation). Via the FOV preview representation, a user may view a projected 3D volume FOV and isocenter on acquired 2D scout images, prior to the imaging system acquiring a 3D image corresponding to the projected 3D volume FOV. Via the FOV preview representation, the imaging system may display to users how movement of the imaging system correlates to the patient anatomy captured in the 2D scout image.

In some aspects, in response to user adjustment of the position or orientation of the O-arm, the imaging system may display (via the FOV preview representation) corresponding changes in isocenter without acquiring another image. In some cases, though the FOV preview feature enables a user to adjust the isocenter of a 3D image volume to be captured in relation to 2D scout images, the user may be unable to fully determine if the desired patient anatomy will be captured in the 3D image volume unless another 2D scout image including the desired patient anatomy is also captured.

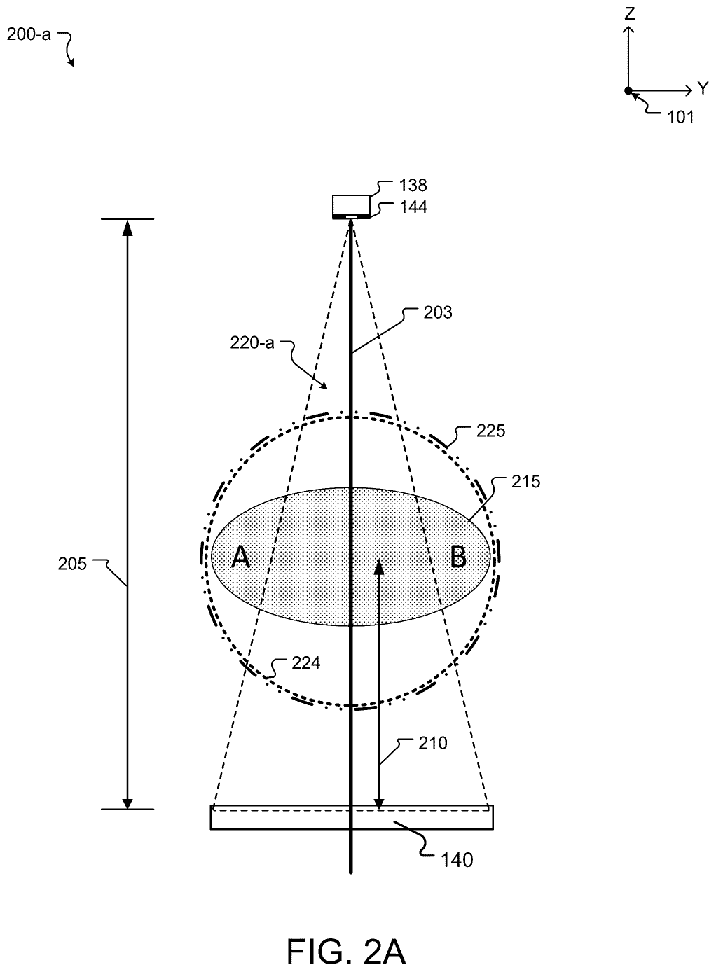

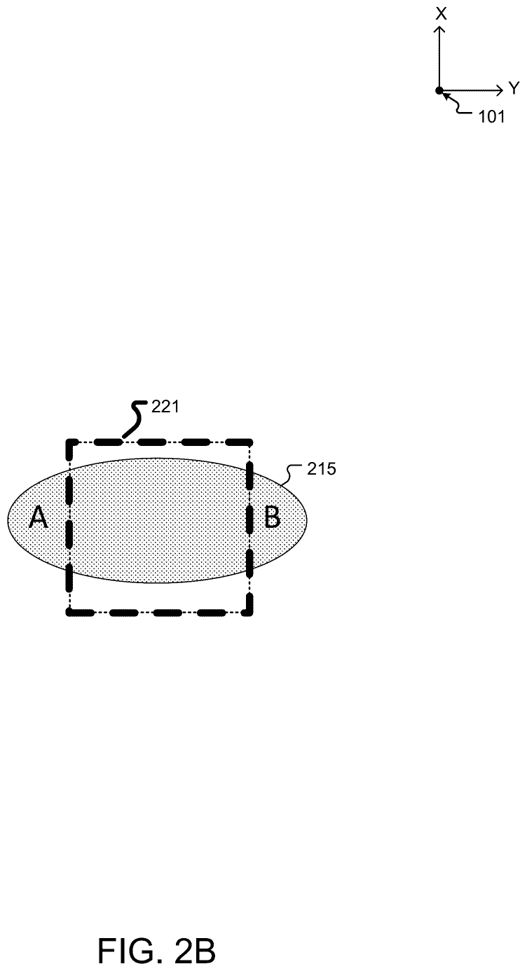

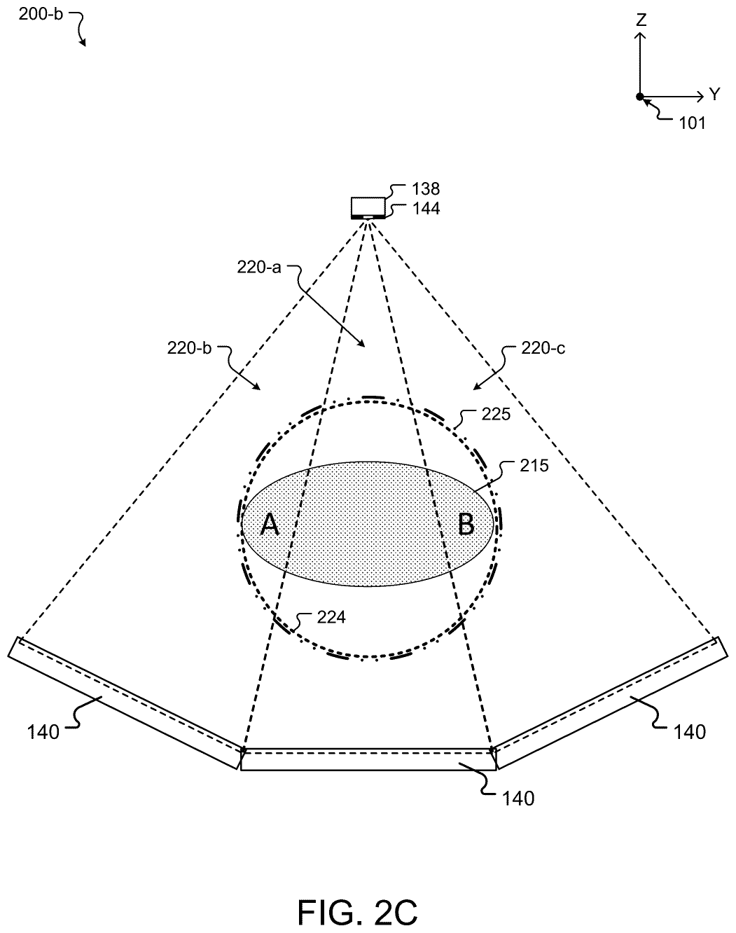

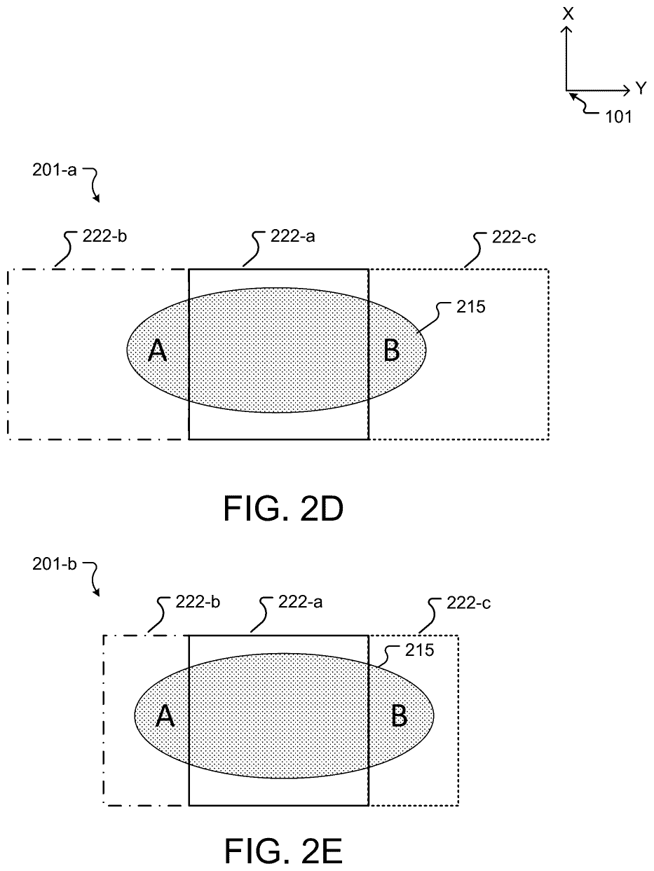

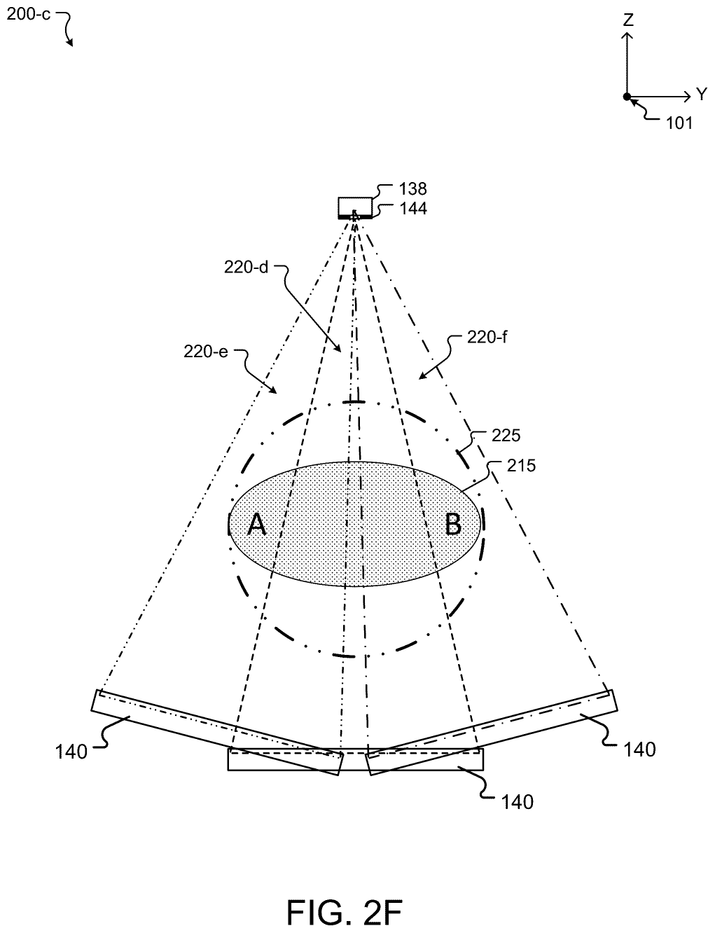

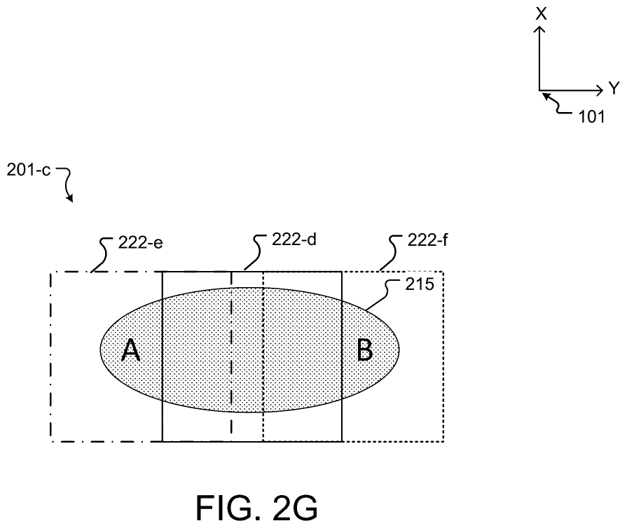

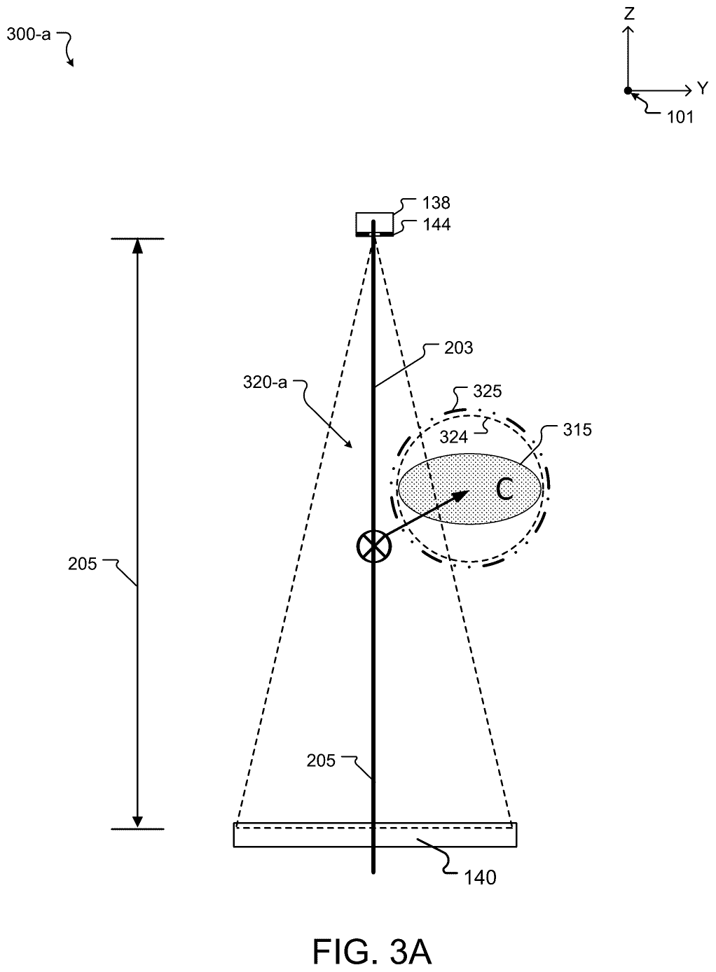

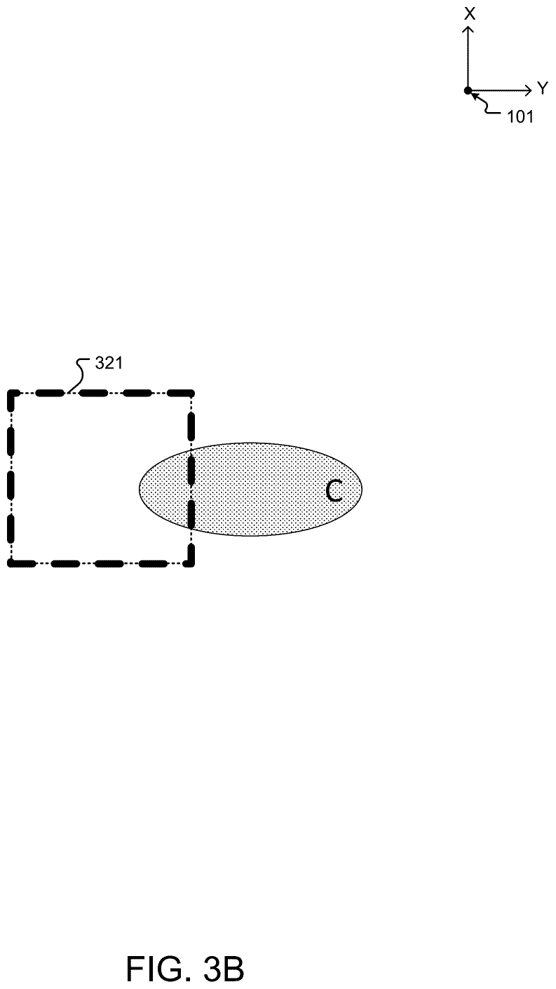

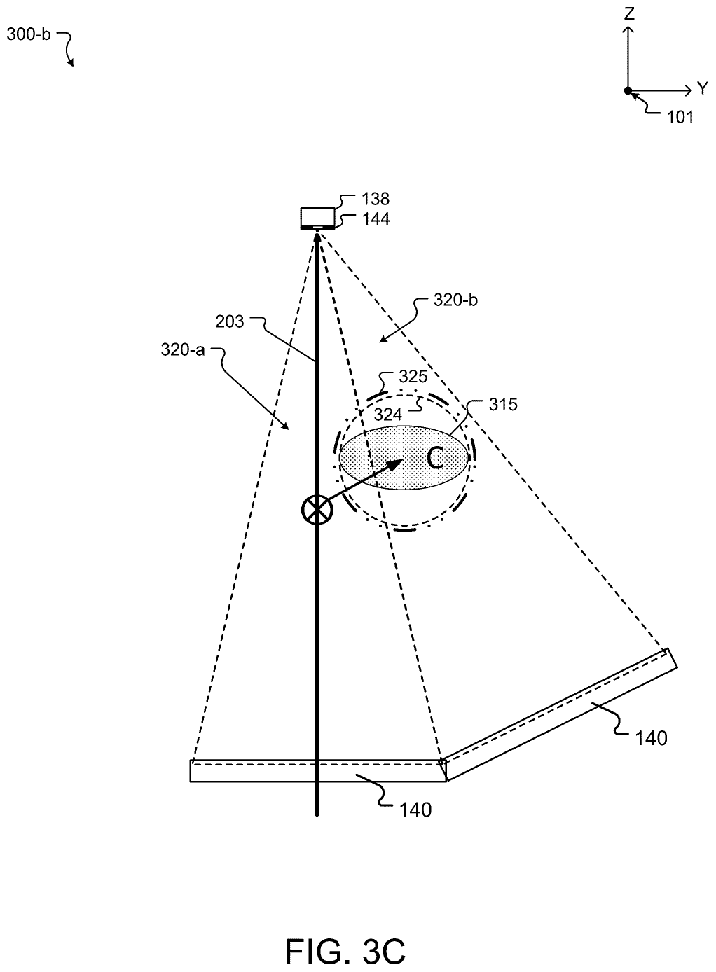

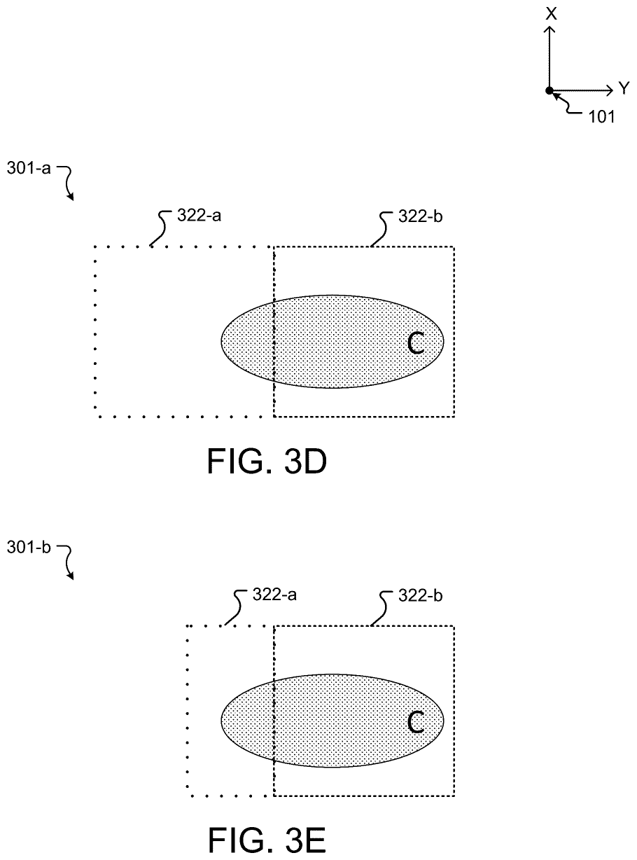

Such cases may result due to fixed x-ray beam collimation and fixed source-to-image detector distance (SID) associated with the O-arm. For example, in some cases, a portion of the patient anatomy that is captured in a 3D image volume using the O-arm may fail to be captured in a 2D scout image used for the FOV preview feature (examples of which is later illustrated at FIGS. 2A and 3A). Accordingly, for example, in some cases, implementing an additional step to reacquire the 2D scout image which captures the portion of the patient anatomy may result in increased radiation dose to the patient and increased time associated with the surgical workflow.

Aspects of the present disclosure support enhancements which combine capabilities of a FOV preview feature with high-lateral imaging, which includes capturing an extended localization image of up to 80 cm (e.g., also referred to herein as an ultrawide image, an ultrawide 2D image, a wide 2D image, an extended 2D image, an extended 2D scout image, or a 2D scout image captured according to an extended width mode of capture).

As will be described and illustrated herein, aspects of the present disclosure support generating an extended localization image generated based on two or more localization images, in which a size of the extended localization image is greater than a corresponding size of each localization image at least with respect to one dimension (e.g., X-axis, Y-axis).

In an example, the systems and techniques described herein include displaying the described extended localization image via the FOV preview feature. In some examples, the systems and techniques may support a user input associated with setting, within the extended localization image, target boundaries of the 3D image volume to be captured.

Accordingly, for example, use of the extended localization image may ensure that all desired patient anatomy will be captured in the 3D image volume without applying additional dose to the patient, as the extended localization image includes the portions of the patient anatomy that are capturable in the 3D image volume.

In some aspects, in response to a user input indicating a region (e.g., target coordinates, target boundaries, etc.) on the extended localization image, the imaging system may move to a target location, position components (e.g., source, detector, gantry, etc.) of the imaging system, and/or position the patient such that the imaging system may capture a 3D image volume that includes the region indicated on the extended localization image.

In some aspects, the systems and techniques described herein may support manual system motion, which may include generating and providing guidance information (e.g., movement data, spatial coordinates, etc.) for positioning the imaging system and/or the patient in association with capturing the 3D image volume.

Additionally, or alternatively, the systems and techniques described herein may support automatic movement of the imaging system. The systems and techniques described herein may incorporate automatic movement, aspects of which are described herein. In an example implementation, for automatic motion, the systems and techniques described herein may include combining the boundary defined on the extended localization image with image orientation information and known system geometry associated with the imaging system. The systems and techniques may include translating the user input into system positioning commands and source-detector acquisition motion.

According to example aspects of the present disclosure, the combination of the FOV preview capability and extended localization image may provide anatomical information that may assist a user in association with localizing target anatomical structures with minimal radiation dose. Accordingly, for example, the techniques described herein may minimize patient and operator risk and increase safety associated with the use of an imaging system, while improving user confidence that patient anatomy for a surgical procedure will be captured in a 3D image volume. In some examples, aspects of the present disclosure support using the localization provided by the extended localization image in conjunction with multiple imaging types (e.g., X-ray imaging, computed tomography (CT) imaging, magnetic resonance imaging (MRI), ultrasound imaging, optical imaging, light detection and ranging (LiDAR) imaging, camera images from an O-arm, preoperative images, intraoperative images, etc.).

Aspects of the extended localization images described herein combined with FOV preview may support various implementations. For example, the systems and techniques described herein may support use cases (e.g., stereotactic cranial procedures, hip procedures, etc.) where scout images having an increased width or increased FOV are required, examples of which are later illustrated herein. The systems and techniques described herein may support use cases of positioning for 3D image volume acquisitions with virtual isocenter shift, examples of which are later illustrated herein. The systems and techniques described herein may support use cases of positioning for 2D long films containing femoral heads and/or the pelvis, examples of which are later illustrated herein. The systems and techniques described herein may support use cases of positioning for 2D long films of severe coronal or sagittal curvature, examples of which are later illustrated herein.

According to example aspects of the present disclosure, the systems and techniques described herein provide features which support increased speed and efficiency associated with 2D image anatomy localization. The features described herein may support increased success associated with 3D volume capture visualization on 2D scout images.

Implementations of the present disclosure provide technical solutions to one or more of the problems of radiation exposure to operators, surgeons, and patients. X-ray exposure can be quantified by dose, or the amount of energy deposited by radiation in tissue. Ionizing radiation can cause debilitating medical conditions. The techniques described herein of combining the FOV preview capability and extended 2D scout images may reduce the risk of capturing a 3D image volume which fails to capture an entire anatomy of interest, which may reduce the risk of additional radiation exposure associated with capturing additional 2D scout images for localizing the patient anatomy and recapturing the 3D image volume. The techniques described herein may allow operators of an imaging system (e.g., an O-arm system) to complete operations of a radiological imaging workflow with increased efficiency and speed.

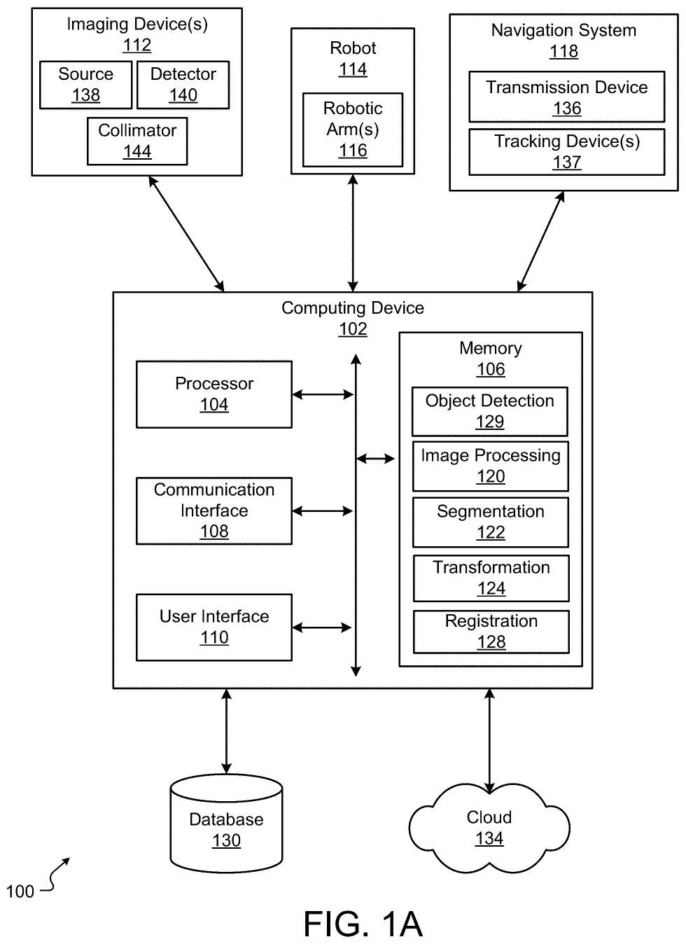

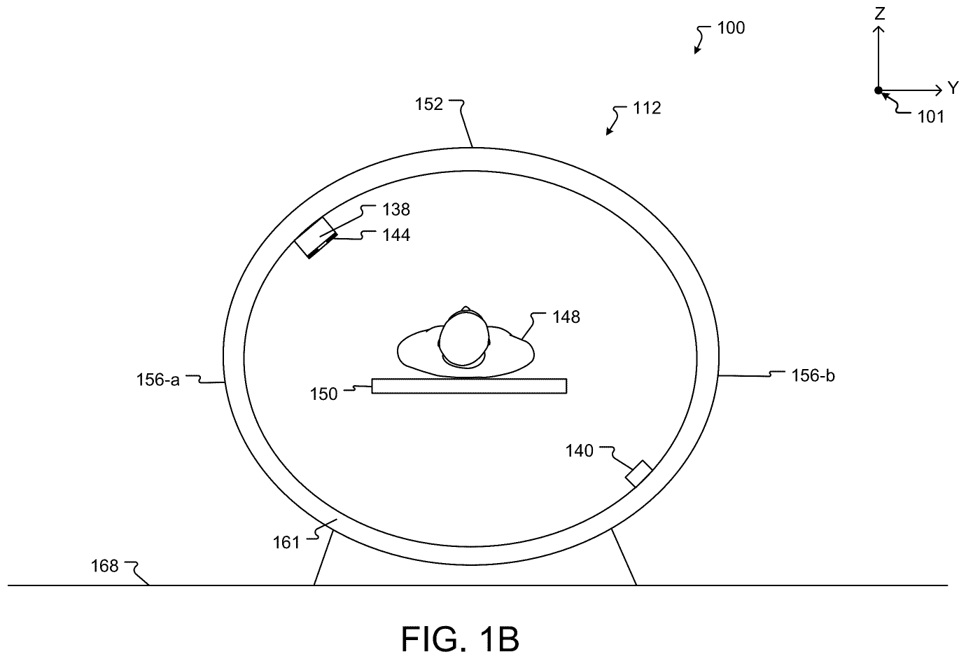

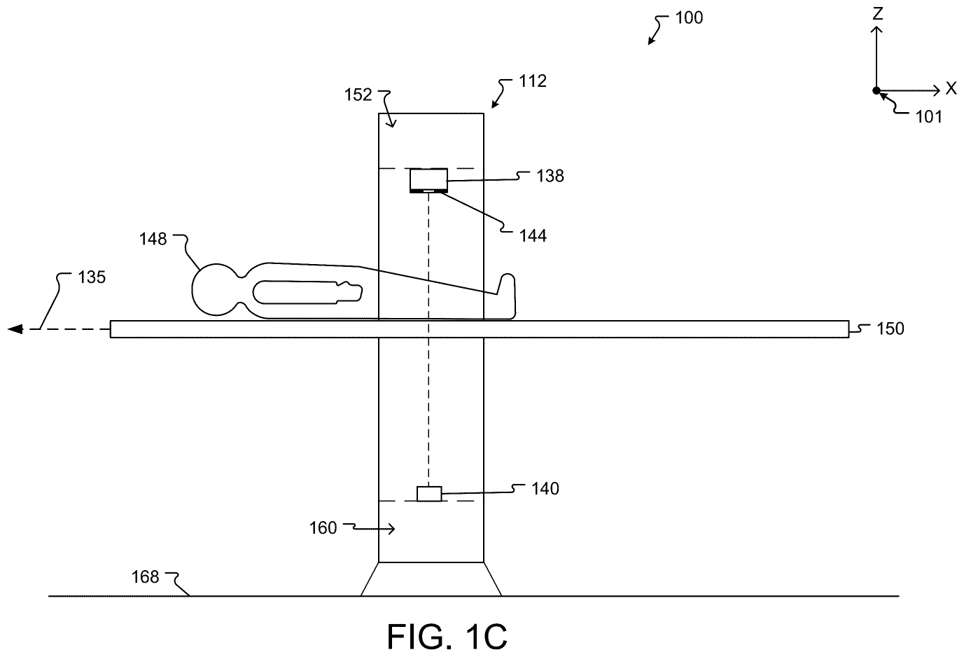

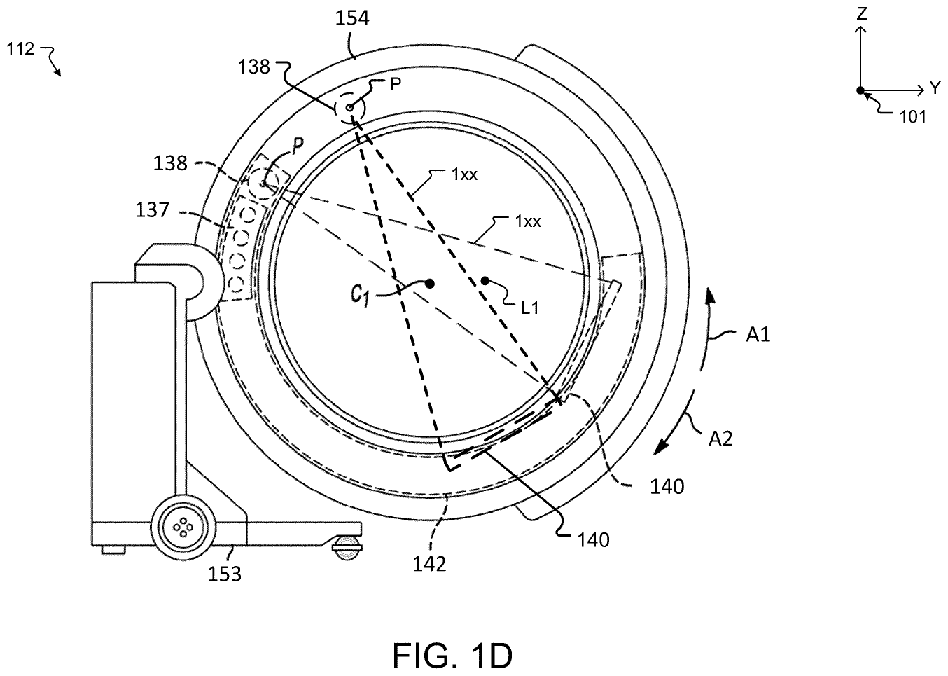

FIGS. 1A through 1D illustrate examples of a system 100 that support aspects of the present disclosure.

Referring to FIG. 1A, the system 100 includes a computing device 102, one or more imaging devices 112, a robot 114, a navigation system 118, a database 130, and/or a cloud network 134 (or other network). Systems according to other implementations of the present disclosure may include more or fewer components than the system 100. For example, the system 100 may omit and/or include additional instances of one or more components of the computing device 102, the imaging device(s) 112, the robot 114, navigation system 118, the database 130, and/or the cloud network 134. In an example, the system 100 may omit any instance of the computing device 102, the imaging device(s) 112, the robot 114, navigation system 118, the database 130, and/or the cloud network 134. The system 100 may support the implementation of one or more other aspects of one or more of the methods disclosed herein.

The computing device 102 includes a processor 104, a memory 106, a communication interface 108, and a user interface 110. Computing devices according to other implementations of the present disclosure may include more or fewer components than the computing device 102. The computing device 102 may be, for example, a control device including electronic circuitry associated with controlling any of the imaging device 112, the robot 114, and the navigation system 118.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging devices 112, the robot 114, the navigation system 118, the database 130, and/or the cloud network 134.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the process flows 300 and 400 described herein, or of any other methods. The memory 106 may store, for example, instructions and/or machine learning models that support one or more functions of the imaging devices 112, the robot 114, and the navigation system 118. For instance, the memory 106 may store content (e.g., instructions and/or machine learning models) that, when executed by the processor 104, enable image processing 120, segmentation 122, transformation 124, registration 128, and/or object detection 129. Such content, if provided as in instruction, may, in some implementations, be organized into one or more applications, modules, packages, layers, or engines.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging devices 112, the robot 114, the navigation system 118, the database 130, and/or the cloud network 134.

The computing device 102 may also include a communication interface 108. The communication interface 108 may be used for receiving data or other information from an external source (e.g., the imaging devices 112, the robot 114, the navigation system 118, the database 130, the cloud network 134, and/or any other system or component separate from the system 100), and/or for transmitting instructions, data (e.g., image data, etc.), or other information to an external system or device (e.g., another computing device 102, the imaging devices 112, the robot 114, the navigation system 118, the database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may include data corresponding to an anatomical feature of a patient, or to a portion thereof. The image data may be or include a preoperative image, an intraoperative image, a postoperative image, or an image taken independently of any surgical procedure. In some implementations, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or include, for example, an ultrasound scanner (which may include, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may include, for example, a transmitter, a receiver, a processor, and one or more antennae), or any other imaging device 112 suitable for obtaining images of an anatomical feature of a patient 148. The imaging device 112 may be contained entirely within a single housing, or may include a transmitter/emitter and a receiver/detector that are in separate housings or are otherwise physically separated.

In some implementations, the imaging device 112 may include more than one imaging device 112. For example, a first imaging device may provide first image data and/or a first image, and a second imaging device may provide second image data and/or a second image. In still other implementations, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The imaging device 112 may include a source 138, a detector 140, and a collimator 144, example aspects of which are later described with reference to FIGS. 1B, 1C, and 1D.

The robot 114 may be any surgical robot or surgical robotic system. The robot 114 may be or include, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some implementations, the robot 114 may be configured to hold and/or manipulate an anatomical element during or in connection with a surgical procedure. The robot 114 may include one or more robotic arms 116. In some implementations, the robotic arm 116 may include a first robotic arm and a second robotic arm, though the robot 114 may include more than two robotic arms. In some implementations, one or more of the robotic arms 116 may be used to hold and/or maneuver the imaging device 112. In implementations where the imaging device 112 includes two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may include one or more sensors that enable the processor 104 (or a processor of the robot 114) to determine a precise pose in space of the robotic arm (as well as any object or element held by or secured to the robotic arm).

In some implementations, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some implementations, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some implementations, the navigation system 118 may include one or more electromagnetic sensors.

In some aspects, the navigation system 118 may include one or more of an optical tracking system, an acoustic tracking system, an electromagnetic tracking system, a radar tracking system, an inertial measurement unit (IMU) based tracking system, and a computer vision based tracking system. The navigation system 118 may include a corresponding transmission device 136 capable of transmitting signals associated with the tracking type. In some aspects, the navigation system 118 may be capable of computer vision based tracking of objects present in images captured by the imaging device(s) 112.

The navigation system 118 may include tracking devices 137. The tracking devices 137 may include or be provided as sensors (also referred to herein as tracking sensors). The system 100 may support the delivery of tracking information associated with the tracking devices 137 to the navigation system 118. The tracking information may include, for example, data associated with signals (e.g., magnetic fields, radar signals, audio signals, etc.) emitted by a transmission device 136 and sensed by the tracking devices 137.

The tracking devices 137 may communicate sensor information to the navigation system 118 for determining a position of the tracked portions relative to each other and/or for localizing an object (e.g., an instrument, an anatomical element, etc.) relative to an image. The navigation system 118 and/or transmission device 136 may include a controller that supports operating and powering the generation of signals to be emitted by the transmission device 136.

In various implementations, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118.

In some implementations, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The processor 104 may utilize data stored in memory 106 as a neural network. The neural network may include a machine learning architecture. In some aspects, the neural network may be or include one or more classifiers. In some other aspects, the neural network may be or include any machine learning network such as, for example, a deep learning network, a convolutional neural network, a reconstructive neural network, a generative adversarial neural network, or any other neural network capable of accomplishing functions of the computing device 102 described herein. Some elements stored in memory 106 may be described as or referred to as instructions or instruction sets, and some functions of the computing device 102 may be implemented using machine learning techniques.

For example, the processor 104 may support machine learning model(s) which may be trained and/or updated based on data (e.g., training data) provided or accessed by any of the computing device 102, the imaging device 112, the robot 114, the navigation system 118, the database 130, and/or the cloud network 134. The machine learning model(s) may be built and updated by the system 100 based on the training data (also referred to herein as training data and feedback).

In some examples, based on the data, the neural network may generate one or more algorithms (e.g., processing algorithms) supportive of object detection 129.

The database 130 may store information that correlates one coordinate system to another (e.g., imaging coordinate systems, robotic coordinate systems, a patient coordinate system, a navigation coordinate system, etc.). The database 130 may additionally or alternatively store, for example, one or more surgical plans (including, for example, pose information about a target and/or image information about a patient's anatomy at and/or proximate the surgical site, for use by the imaging device 112, robot 114, the navigation system 118, and/or a user of the computing device 102 or of the system 100); one or more images useful in connection with a surgery to be completed or analyzed; and/or any other useful information. The database 130 may additionally or alternatively store, for example, images captured or generated based on image data provided by the imaging device 112.

The database 130 may be configured to provide any such information to the computing device 102 or to any other device of the system 100 or external to the system 100, whether directly or via the cloud network 134. In some implementations, the database 130 may be or include part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VOIP) network, a cellular network, and any other type of packet-switched or circuit-switched network known in the art. In some cases, the communications network may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of any of the methods 300 and/or 400 described herein. The system 100 or similar systems may also be used for other purposes.

FIG. 1B illustrates an example of the system 100 that supports aspects of the present disclosure. FIG. 1C illustrates an example of the system 100 that supports aspects of the present disclosure. FIG. 1D illustrates an example of the system 100 that supports aspects of the present disclosure. Aspects of the system 100 previously described with reference to FIG. 1A and descriptions of like elements are omitted for brevity.

With reference to FIGS. 1B through ID, features of the system 100 may be described in conjunction with a coordinate system 101. The coordinate system 101, as shown in FIGS. 1B, 1C, and 2A through 2D, includes three-dimensions including an X-axis, a Y-axis, and a Z-axis. Additionally or alternatively, the coordinate system 101 may be used to define planes (e.g., the XY-plane, the XZ-plane, and the YZ-plane) of the system 100. These planes may be disposed orthogonal, or at 90 degrees, to one another. While the origin of the coordinate system 101 may be placed at any point on or near the components of the system 100 (e.g., components of the imaging device 112, for the purposes of description, the axes of the coordinate system 101 are always disposed along the same directions from figure to figure, whether the coordinate system 101 is shown or not. In some examples, reference may be made to dimensions, angles, directions, relative positions, and/or movements associated with one or more components of the system 100 (e.g., imaging device 112) with respect to the coordinate system 101.

The system 100 may be used to initiate long scans of a patient, adjust imaging components to capture localization images of the patient, set or identify target coordinates associated with the localization images, perform a long scan process based on the localization images and the target coordinates, capture multidimensional images based on the target coordinates, and generating a long scan image based on the multidimensional images.

The imaging device 112 includes an upper wall or member 152, a lower wall 161 (also referred to herein as member 161), and a pair of sidewalls 156-a and 156-b (also referred to herein as members 156-a and 156-b). In some embodiments, the imaging device 112 is fixed securable to an operating room surface 168 (such as, for example, a ground surface of an operating room or other room). In other embodiments, the imaging device 112 may be releasably securable to the operating room wall 168 or may be a standalone component that is simply supported by the operating room wall 168.

A table 150 configured to support the patient 148 may be positioned orthogonally to the imaging device 112, such that the table 150 extends in a first direction from the imaging device 112. In some embodiments, the table 150 may be mounted to the imaging device 112. In other embodiments, the table 150 may be releasably mounted to the imaging device 112. In still other embodiments, the table 150 may not be attached to the imaging device 112. In such embodiments, the table 150 may be supported and/or mounted to an operating room wall, for example. In embodiments where the table 150 is mounted to the imaging device 112 (whether detachably mounted or permanently mounted), the table 150 may be mounted to the imaging device 112 such that a pose of the table 150 relative to the imaging device 112 is selectively adjustable. The patient 148 may be positioned on the table 150 in a supine position, a prone position, a recumbent position, and the like.

The table 150 may be any operating table configured to support the patient 148 during a surgical procedure. The table 150 may include any accessories mounted to or otherwise coupled to the table 150 such as, for example, a bed rail, a bed rail adaptor, an arm rest, an extender, or the like. The table 150 may be stationary or may be operable to maneuver the patient 148 (e.g., the table 150 may be moveable). In some embodiments, the table 150 has two positioning degrees of freedom and one rotational degree of freedom, which allows positioning of the specific anatomy of the patient anywhere in space (within a volume defined by the limits of movement of the table 150). For example, the table 150 may slide forward and backward and from side to side, tilt (e.g., around an axis positioned between the head and foot of the table 150 and extending from one side of the table 150 to the other) and/or roll (e.g., around an axis positioned between the two sides of the table 150 and extending from the head of the table 150 to the foot thereof). In other embodiments, the table 150 may be bendable at one or more areas (which bending may be possible due to, for example, the use of a flexible surface for the table 150, or by physically separating one portion of the table 150 from another portion of the table 150 and moving the two portions independently). In at least some embodiments, the table 150 may be manually moved or manipulated by, for example, a surgeon or other user, or the table 150 may include one or more motors, actuators, and/or other mechanisms configured to enable movement and/or manipulation of the table 150 by a processor such as a processor 104 of the computing device 102.

The imaging device 112 includes a gantry. The gantry may be or include a substantially circular, or “O-shaped,” housing that enables imaging of objects placed into an isocenter thereof. In other words, the gantry may be positioned around the object being imaged. In some embodiments, the gantry may be disposed at least partially within the member 152, the sidewall 156-a, the sidewall 156-b, and the lower wall 161 of the imaging device 112.

The imaging device 112 also includes a source 138 and a detector 140. The source 138 may be a device configured to generate and emit radiation, and the detector 140 may be a device configured to detect the emitted radiation. In some embodiments, the source 138 and the detector 140 may be or include an imaging source and an imaging detector (e.g., the source 138 and the detector 140 are used to generate data useful for producing images). The source 138 may be positioned in a first position and the detector 140 may be positioned in a second position opposite the source 138. In some embodiments, the source 138 may include an X-ray source (e.g., a thermionic emission tube, a cold emission x-ray tube, or the like). The source 138 may project a radiation beam that passes through the patient 148 and onto the detector 140 located on the opposite side of the imaging device 112. The detector 140 may be or include one or more sensors that receive the radiation beam (e.g., once the radiation beam has passed through the patient 148) and transmit information related to the radiation beam to one or more other components (e.g., processor 104) of the system 100 for processing.

In some embodiments, the detector 140 may include an array. For example, the detector 140 may include three 2D flat panel solid-state detectors arranged side-by-side, and angled to approximate the curvature of the imaging device 112. It will be understood, however, that various detectors and detector arrays can be used with the imaging device 112, including any detector configurations used in typical diagnostic fan-beam or cone-beam CT scanners. For example, the detector 140 may include a 2D thin-film transistor X-ray detector using scintillator amorphous-silicon technology.

The source 138 may be or include a radiation tube (e.g., an x-ray tube) capable of generating the radiation beam. In some embodiments, the source 138 and/or the detector 140 may include a collimator 144 configured to confine or shape the radiation beam emitted from the source 138 and received at the detector 140. Once the radiation beam passes through patient tissue and received at the detector 140, the signals output from the detector 140 may be processed by the processor 104 to generate a reconstructed image of the patient tissue. In this way, the imaging device 112 can effectively generate reconstructed images of the patient tissue imaged by the source 138 and the detector 140.

The source 138 and the detector 140 may be attached to the gantry and configured to rotate 360 degrees around the patient 148 in a continuous or step-wise manner so that the radiation beam can be projected through the patient 148 at various angles. In other words, the source 138 and the detector 140 may rotate, spin, or otherwise revolve about an axis that passes through the top and bottom of the patient 148, with the patient anatomy that is the subject of the imaging positioned at the isocenter of the imaging device 112. The rotation may occur through a drive mechanism that causes the gantry to move such that the source 138 and the detector 140 encircle the patient 148 on the table 150.

At each projection angle, the radiation beam passes through and is attenuated by the patient 148. The attenuated radiation is then detected by the detector 140. The detected radiation from each of the projection angles can then be processed, using various reconstruction techniques, to produce a 2D or 3D reconstruction image of the patient 148. For example, the processor 104 may be used to perform image processing 120 to generate the reconstruction image. Additionally or alternatively, the source 138 and the detector 140 may move along a length of the patient 148, as depicted in FIG. 1B. For example, the table 150 holding the patient 148 may move in the direction of arrow 135 while the source 138 and detector 140 remain in a fixed location, such that the length of the patient can be scanned. In such embodiments, the scanned data may be used to generate one or more reconstructed images of the patient 148 and/or a long scan image of the patient 148.

The imaging device 112 may be included in the O-Arm® imaging system sold by Medtronic Navigation, Inc. having a place of business in Louisville, Colo., USA. The O-Arm® imaging system can include a mobile cart 153 (later illustrated at FIG. 1D) that supports movement of the imaging device 112 from one operating theater or room to another, and the gantry may move relative to the mobile cart 153.

In some example implementations, the system 100 may perform a long scan process by moving the source 138 and the detector 140 along a direction of an axis running through the patient 148. In some embodiments, the direction of the axis along which the source 138 and the detector 140 move may be the same direction as (e.g., extend in a direction parallel to) the direction indicated by the arrow 135. In some embodiments, the long scan process may include moving the patient 148 relative to the source 138 and the detector 140. For example, the patient 148 may be positioned in a prone position on the table 150. The system 100 may move the table 150 through an isocenter of the imaging device 112, in the direction (or opposite the direction) indicated by the arrow 135, such that the source 138 and the detector 140 generate projection data along a length of the patient 148.

In some embodiments, the system 100 may move the source 138 and the detector 140 relative to the patient 148 (or move the table 150 and patient 148 relative to the source 138 and the detector 140) at a predetermined rate and/or a fixed rate.

Referring to FIG. 1D, the imaging device 112 can include a tracking device 137 (e.g., an optical tracking device, an electromagnetic tracking device, etc.) to be tracked by the navigation system 118. The tracking device 137 can be associated directly with the source 138, the detector 140, rotor 142, the gantry 154, or other appropriate part of the imaging device 112 to determine the location or position of the source 138, detector 140, rotor 142, and/or gantry 154 relative to a selected reference frame. As illustrated, the tracking device 137 can be positioned on the exterior of the housing of the gantry 154. Accordingly, the imaging device 112 can be tracked relative to the patient 148 as can an instrument (not illustrated) in association with initial registration, automatic registration, or continued registration of the patient 148 relative to the image data.

With reference to FIG. 1D, the gantry 154 may define an isocenter of the imaging device 112. For example, a centerline C1 (represented as a dot on the ZY plane of the coordinate system 101) through the gantry 154 may define an isocenter or center of the imaging device 112, and any other line through the gantry 154, such as L1 (represented as a dot on the ZY plane of the coordinate system 101), may be considered to be off-isocenter or off-center of the imaging device 112. With reference to FIG. 1D, the patient 148 may be positioned along the centerline CI of the gantry 154, such that a longitudinal axis (extending parallel to the X axis of the coordinate system 101) of the patient 148 is aligned with the isocenter of the imaging device 112. Image data acquired along the centerline C1 of the imaging device 112 may be considered isocenter or center image data, and image data acquired off-isocenter or off-center may be considered off-isocenter or off-center image data.

With reference to FIG. 1D, the source 138 may emit x-rays toward the patient 148, and the x-rays may pass through the patient and be detected by the detector 140. As is understood by one skilled in the art, the x-rays emitted by the source 138 may be emitted in a cone (a cone shape) and detected by the detector 140. The source 138 and the detector 140 may each be coupled to the rotor 142 so as to be generally diametrically opposed within the gantry 154, and movable within the gantry 154 about the patient 148. The detector 140 may move rotationally in a 360° motion around the patient 148 generally in the directions of arrows A1 and A2, and the source 138 may move in concert with the detector 140 such that the source 138 remains about 180° apart from and opposed to the detector 140.

The source 138 may be pivotably mounted to the rotor 142 and controlled by an actuator, such that the source 138 may be controllably pivoted about a focal spot P of the source 138 relative to the rotor 142 and the detector 140. By controllably pivoting the source 138, the system 100 may angle or alter the trajectory of the x-rays relative to the patient 148, without repositioning the patient 148 relative to the gantry 154. Further, the detector 140 may move about an arc relative to the rotor 142, in the direction of arrows A1 and A2. In one example, the detector 140 may pivot about the focal spot P of the source 138, such that the source 138 and detector 140 may pivot about the same angle. As the detector 140 may pivot at the same angle as the source 138, the detector 140 may detect the x-rays emitted by the source 138 at any desired pivot angle, which may enable the acquisition of off-center image data, examples of which will be discussed further herein. The rotor 142 may be rotatable about the gantry 154 in association with acquiring target image data (on center or off-center).

In some aspects, the system 100 may support an extended width mode of capture, which may include moving the source 138 and the detector 140 relative to the patient 148 in association with capturing multiple 2D localization images and generating, from the multiple 2D localization images, an extended localization image.

FIG. 2A illustrates an example 200-a of a beam geometry corresponding to an x-ray field 220-a emitted from the source 138 and collimator 144 (collimator window) of an imaging device 112, as supported by the system 100 in accordance with aspects of the present disclosure. The imaging device 112 may emit the x-ray field 220-a in association with capturing a localization image 221.

As illustrated at FIG. 2A, the imaging device 112 may have a fixed x-ray beam collimation and SID 205, and the imaging device 112 may support capturing a localization image 221 (illustrated at FIG. 2B) according to a non-extended width mode of capture, in which a source to object distance (SOD) 210 between the detector 140 and an object 215 (e.g., a patient anatomy) is constant. An example case is described herein with reference to capturing the localization image 221 according to the non-extended width mode, and further capturing an image volume 225 (also referred to herein as a 3D large FOV and 3D extended FOV).

The imaging device 112 may display the localization image 221 via a FOV preview representation. As illustrated at FIG. 2B, the localization image 221 does not include image data corresponding to regions A and B of the object 215. For example, regions A and B are cutoff from the localization image 221.

Accordingly, for example, the localization image 221 may generate a projected image volume 224 corresponding to the image volume 225. The imaging device 112 may display, in the FOV preview representation, the projected extended image volume 224. In the example of FIGS. 2A and 2B, portions of the projected image volume 224 corresponding to regions A and B of the object 215 may be absent any image data corresponding to regions A and B, as the localization image 221 does not include image data corresponding to regions A and B of the object 215.

Since regions A and B were not captured in the localization image 221 (and accordingly, for example, in the projected image volume 224), the user would be unable to fully determine whether regions A and B would be captured in the image volume 225 unless additional localization images 221 that include regions A and B are captured.

In another example, the user may control the imaging device 112 (e.g., set motion parameters associated with controlling or positioning the imaging device 112, etc.) for capturing the image volume 225 such that the image volume 225 would include a target patient anatomy (e.g., object 215 and regions A and B of the object 215). However, in some cases, after the imaging device 112 captures the image volume 225, the user may discover that the image volume 225 does not include the regions A and B.

As will be described with reference to FIGS. 2C through 2G, aspects of the present disclosure include generating an extended localization image 201 that further captures areas A and B, which may eliminate instances of further acquiring additional localization images (e.g., for capturing areas A and B not captured in the localization image 221) or reacquiring the image volume 225, which would otherwise subject the patient 148 to increased radiation dose and increase time associated with the surgical workflow. The systems and techniques described herein may prevent instances in which 1) a projected image volume 224 does not include image data corresponding to a target patient anatomy (e.g., object 215 and regions A and B of the object 215) and/or 2) an image volume 225, as captured by the imaging device 112, does not include the target patient anatomy.

FIG. 2C illustrates an example 200-b of generating an extended localization image 201 (also referred to herein as a fused localization image) that captures object 215, including regions A and B that are outside of the x-ray field 220-a.

In an example, with reference to FIGS. 2C and 2D, the imaging device 112 may capture multiple localization images 222 (e.g., localization image 222-a through localization image 222-c) in association with a scan process according to an extended width mode of capture. In an example, the imaging device 112 may capture the multiple localization images 222 based on respective positions or orientations of components (e.g., source 138, collimator 144, detector 140, etc.) of the imaging device 112.

In an example, the imaging device 112 may respectively capture localization image 222-a, localization image 222-b, and localization image 222-c based on x-ray field 220-a, x-ray field 220-b, and x-ray field 220-c. It is to be understood that x-ray field 220-a through x-ray field 220-c may refer to the same x-ray field as generated by the source 138, but as emitted based on different positions or orientations of the source 138 (and/or collimator 144).

The system 100 may generate an extended localization image 201 (e.g., extended localization image 201-a, extended localization image 201-b, etc.) including the entirety of the object 215, including regions A and B, based on image data of the localization image 222-a through localization image 222-c. In an example, the system 100 may merge the image data from the localization image 222-a through localization image 222-c (e.g., capture and “stitch” together the multidimensional images) in association with generating the extended localization image 201.

Referring to the example of FIG. 2D, in some example implementations, the extended localization image 201-a includes the entirety of localization image 222-a through localization image 222-c.

Referring to the example of FIG. 2E, in some other example implementations, the system 100 may omit portions of image data of any of the localization images 222. For example, the extended localization image 201-b includes localization image 222-a, a portion (e.g., half) of localization image 222-b, and a portion (e.g., half) of localization image 222-c.

In an example, the extended localization image 201-b may have a width (e.g., with respect to the Y-axis of coordinate system 101) of about 80 centimeters. For example, the localization image 222-a may have a width of about 40 cm FOV at a geometric isocenter 203 of the imaging device 112, the localization image 222-b may have a width of about 20 cm, and the localization image 222-c may have a width of about 20 cm.

It is to be understood that the systems and techniques support capturing overlapping or non-overlapping localization images. For example, image data of localization image 222-a may overlap with image data of localization image 222-b and/or image data of localization image 222-c with respect to an axis (e.g., Y-axis).

In an example implementation, referring to FIGS. 2C, 2D, and 2E, the system 100 may generate a projected image volume 224 (corresponding to the image volume 225) from the image data of an extended localization image 201 (e.g., extended localization image 201-a, extended localization image 201-b). The projected image volume 224 may include volume data including the object 215 (including regions A and B of the object 215). Based on the inclusion of regions A and B in the projected image volume 224, the user would be able to confirm that the system 100 would capture regions A and B in the image volume 225.

It is to be understood that, although illustrated as different sizes at FIG. 2C, the projected image volume 224 may be equal in size to the image volume 225. For simplicity, the projected image volume 224 is not illustrated in the later example described with reference to FIG. 2F.

FIG. 2F illustrates an example 200-c of generating an extended localization image 201-c (illustrated at FIG. 2G) in which captured localization images 222-d through localization image 222-f overlap. As illustrated at FIG. 2F, positions of the detector 140 overlap in association with capturing localization images 222-d through localization image 222-f, and x-ray field 220-d partially overlaps with x-ray field 220-c and x-ray field 220-f.

It is to be understood that aspects of the present disclosure support various widths for the extended localization images 201 and the localization images 222, and the example dimensions of the extended localization images 201 and the localization images 222 as described herein are not limited thereto.

FIG. 3A illustrates an example 300-a of a beam geometry corresponding to an x-ray field 320-a emitted from the source 138 and collimator 144 of the imaging device 112, as supported by the system 100 in accordance with aspects of the present disclosure. The imaging device 112 may emit x-ray field 220-d in association with capturing a localization image 321 of an object 315. An example case is described with reference to capturing the localization image 321 according to a non-extended width mode of capture, and further capturing an image volume 325 (also referred to herein as a 3D large FOV).

The imaging device 112 may display the localization image 321 via a FOV preview representation. As illustrated at FIG. 3B, the localization image 321 does not include image data corresponding to region C of the object 215. For example, region C is cutoff from the localization image 321.

Accordingly, for example, the imaging device 112 may generate a projected image volume 324 corresponding to the image volume 325. The imaging device 112 may display, in the FOV preview representation, the projected image volume 324. In the example of FIG. 3A, the projected image volume 324 is a virtual isocenter shifted 3D image FOV. In the example, a portion of the projected image volume 324 corresponding to region C of the object 315 may be absent any image data corresponding to region C, as the localization image 321 does not include image data corresponding to region C.

Since region C was not captured in the localization image 321 (and accordingly, for example, in the projected image volume 324), the user would be unable to fully determine, from the projected image volume 324, whether region C would be captured in the image volume 325 unless an additional localization image 321 that includes region C is captured.

In another example, the user may control the imaging device 112 (e.g., set motion parameters associated with controlling or positioning the imaging device 112, etc.) for capturing the image volume 325 such that the image volume 325 would include a target patient anatomy (e.g., object 315 and region C of the object 315). However, in some cases, after the imaging device 112 captures the image volume 325, the user may discover that the image volume 325 does not include the region C.

As will be described with reference to FIGS. 3C through 3E, aspects of the present disclosure include generating an extended localization image 301 that further captures region C, which may eliminate instances of further acquiring additional localization images (e.g., for capturing region not captured in the localization image 321) or reacquiring the image volume 325, which would otherwise subject the patient 148 to increased radiation dose and increase time associated with the surgical workflow. The systems and techniques described herein may prevent instances in which 1) a projected image volume 324 does not include image data corresponding to a target patient anatomy (e.g., region C of the object 315) and/or 2) an image volume 325, as captured by the imaging device 112, does not include the target patient anatomy.

The examples described with reference to FIGS. 3C and 3D may include aspects of the system 100 as described with reference to FIGS. 2A through 2G, and descriptions of like elements are omitted for brevity.

FIG. 3C illustrates an example 300-b of generating an extended localization image 301 (also referred to herein as a fused localization image) that captures object 315, including region C that is outside of the x-ray field 320-a.

In an example, with reference to FIGS. 3C and 3D, the imaging device 112 may capture multiple localization images 322 (e.g., localization image 322-a and localization image 322-b) in association with a scan process according to an extended width mode of capture. In an example, the imaging device 112 may capture the multiple localization images 322 based on respective positions or orientations of components (e.g., source 138, collimator 144, detector 140, etc.) of the imaging device 112.

In an example, the imaging device 112 may respectively capture localization image 322-a and localization image 322-b based on x-ray field 320-a and x-ray field 320-b. It is to be understood that x-ray field 320-a and x-ray field 320-b may refer to the same x-ray field as generated by the source 138, but as emitted based on different positions or orientations of the source 138 (and/or collimator 144).

The system 100 may generate an extended localization image 301 (e.g., extended localization image 301-a, extended localization image 301-b, etc.) including the entirety of the object 315, including region C, based on image data of the localization image 322-a and localization image 322-b. In an example, the system 100 may merge the image data from the localization image 322-a and localization image 322-b (e.g., capture and “stitch” together the multidimensional images) in association with generating the extended localization image 301.

Referring to the example of FIG. 3D, in some example implementations, the extended localization image 301-a includes the entirety of localization image 322-a and localization image 322-b.

In an example, the extended localization image 301-a may have a width (e.g., with respect to the Y-axis of coordinate system 101) of about 80 centimeters. For example, the localization image 322-a may have a width of about 40 cm FOV at the geometric isocenter 203 of the imaging device 112, and the localization image 322-b may have a width of about 40 cm.

Referring to the example of FIG. 3E, in some other example implementations, the system 100 may omit portions of image data of any of the localization images 322. For example, the extended localization image 301-b includes localization image 322-b and a portion of localization image 322-a.

It is to be understood that the systems and techniques support capturing overlapping or non-overlapping localization images. For example, image data of localization image 322-a may overlap with image data of localization image 322-b with respect to an axis (e.g., Y-axis). For example, positions of the detector 140 may overlap in association with capturing localization image 322-a and localization image 322-b, and x-ray field 320-a may partially overlap with x-ray field 320-b.

In an example implementation, referring to FIGS. 3C, 3D, and 3E, the system 100 may generate a projected image volume 324 (corresponding to the image volume 325) from the image data of an extended localization image 301 (e.g., extended localization image 301-a, extended localization image 301-b). The projected image volume 324 may include volume data including the object 315 (including region C of the object 315). Based on the inclusion of region C in the projected image volume 324, the user would be able to confirm that the system 100 would capture region C in the image volume 325. It is to be understood that, although illustrated as different sizes at FIG. 3C, the projected image volume 324 may be equal in size to the image volume 325.

It is to be understood that aspects of the present disclosure support various widths for the extended localization images 301 and the localization images 322, and the dimensions of the extended localization images 301 and the localization images 322 as described herein are not limited thereto.

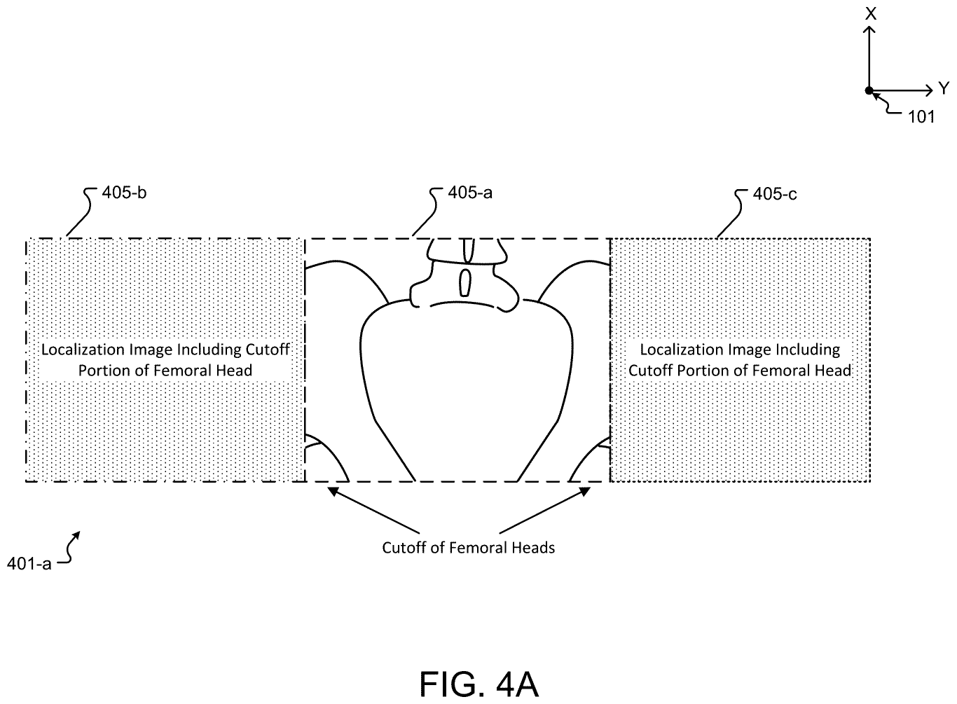

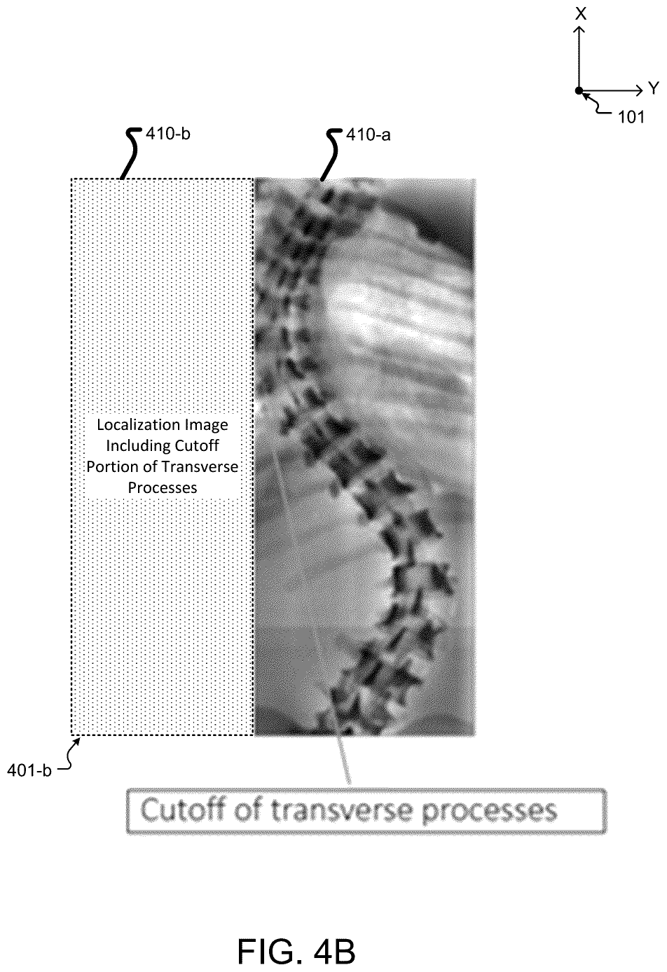

FIG. 4A illustrates an example of a localization image 405-a in which femoral heads extend beyond a fixed collimated 2D FOV of the localization image 405-a. The systems and techniques described herein support capturing localization image 405-a through localization image 405-c, and further, generating an extended localization image 401-a by merging the image data of the localization image 405-a through localization image 405-c as described in accordance with aspects of the present disclosure. Portions of the femoral heads that are cutoff from the localization image 405-a are included in the localization image 405-b and localization image 405-c, and accordingly, for example, are included the 2D FOV of the extended localization image 401-a.

The system 100 may display the extended localization image 401-a via a user interface 110. The systems and techniques described herein may support user selection of a target associated with the extended localization image 401-a. The target may include, for example, a target object, a target region, target coordinates, a target center, and the like associated with the extended localization image 401-a. Based on the target associated with the extended localization image 401-a, the system 100 may position components of the imaging device 112 for a subsequent scan (e.g., capturing a 2D long film image including the femoral heads and/or the pelvis, capturing an image volume including the femoral heads and/or the pelvis as described herein, etc.).

The system 100 may support AI and computer vision based selection of the target for the extended localization image 401-a. For example, the system 100 may detect a target feature (e.g., femoral heads) using object detection 129 described with reference to FIG. 1A. The system 100 may display an indicator (e.g., highlighting, outline, etc.) corresponding to the target feature and/or an indicator (e.g., a circle, a dotted circle, a rectangle, etc.) corresponding to a target region, a target center, or the like. In some aspects, the system 100 may set candidate target coordinates in association with the target feature.

In some examples, the system 100 may alert the user of the target feature included in the extended localization image 401-a and the candidate target coordinates. In some aspects, the system 100 may support features for user confirmation (e.g., approval, denial) and user modification of the candidate target coordinates. In some example implementations the system 100 may identify and/or set the target coordinates without a user input.

FIG. 4B illustrates an example of a localization image 410-a of a spinal structure of a patient 148, in which a coronal deformity extends beyond a fixed collimated 2D FOV of the localization image 410-a. For example, transverse processes of the spinal structure are cut off from the localization image 410-a. The systems and techniques described herein support capturing localization image 410-a and localization image 410-b, and further, generating an extended localization image 401-b by merging the image data of the localization image 410-a and localization image 410-b. Portions of the coronal deformity (e.g., transverse processes) that are cutoff from the localization image 410-a are included in the localization image 410-b, and accordingly, for example, are included the 2D FOV of the extended localization image 401-b.

The example aspects described herein with reference to FIG. 4A may be applied to the example of FIG. 4B. For example, the system 100 may support displaying the extended localization image 401-b via user interface 110, user selection of a target (e.g., coronal deformity, sagittal curvature, etc.) associated with the extended localization image 401-b, AI and computer vision based selection of the target, and positioning components of the imaging device 112 for a subsequent scan (e.g., capturing a 2D long film image including portions of the target, capturing an image volume including portions of the target as described herein, etc.).

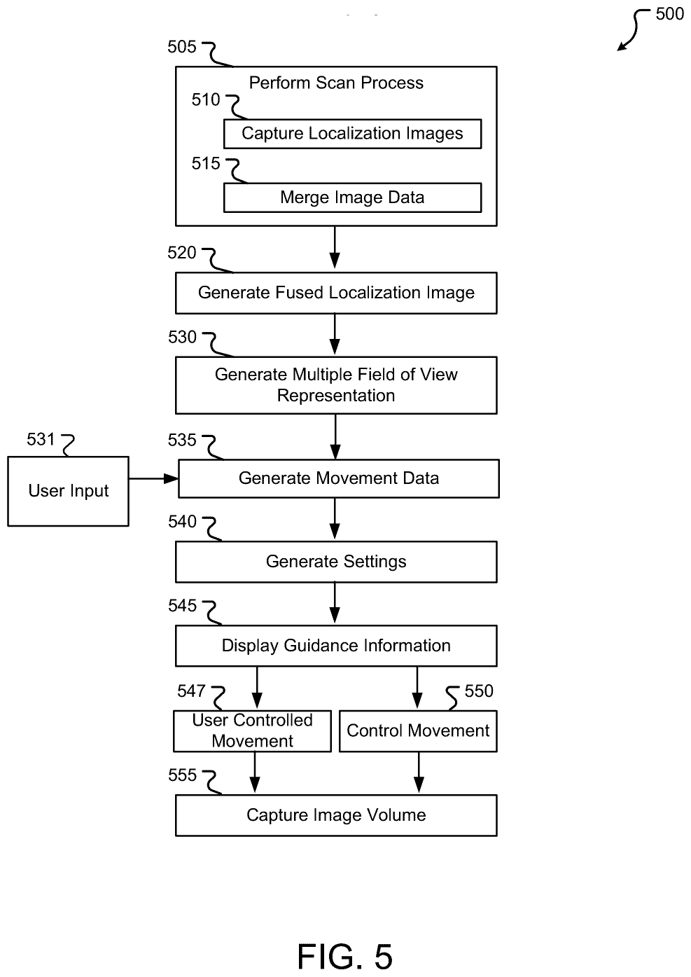

FIG. 5 illustrates an example of a process flow 500 in accordance with aspects of the present disclosure. In some examples, process flow 500 may be implemented by aspects of the system 100 described herein.

In the following description of the process flow 500, the operations may be performed in a different order than the order shown, or the operations may be performed in different orders or at different times. Certain operations may also be left out of the process flow 500, or other operations may be added to the process flow 500.

It is to be understood that any device (e.g., computing device 102, imaging device 112, etc.) of the system 100 may perform the operations shown.

The process flow 500 is described herein with reference to FIGS. 1A through ID and FIGS. 2A through 2G, and FIGS. 3A through 3E.

At 505, the system 100 may perform a scan process of a patient anatomy (e.g., object 215, object 315, etc.) according to an extended width mode of capture.

In some aspects, in performing the scan process according to the extended width mode of capture, the system 100 may capture (at 510) localization images (e.g., localization images 222, localization images 322) associated with the patient anatomy.

At 515, the system 100 may merge image data associated with the localization images.

At 520, the system 100 may generate a fused localization image (e.g., extended localization image 201, extended localization image 301, etc.) of the patient anatomy based on the merged image data.

In some aspects, boundaries of the fused localization image (e.g., extended localization image 201, extended localization image 301, etc.) may correspond to one or more boundaries of the localization images (e.g., localization images 222, localization images 322, etc.). For example, referring to FIGS. 2D and 2E, boundaries of the extended localization images 201-a and 201-b may be based on boundaries of localization images 222. In another example, In another example, referring to FIG. 3D, boundaries of the extended localization images 301-a and 301-b may be based on boundaries of localization images 322.

At 530, the system 100 may generate a multiple FOV representation (e.g., FOV preview representation) of the patient anatomy based on target boundaries associated with the fused localization image, image data included in the fused localization image, and an image volume (e.g., image volume 225, image volume 325) to be captured. In some aspects, the multiple FOV representation includes a projected image volume (e.g., projected image volume 224, projected image volume 324, etc.) as described herein.

In an example, via the multiple FOV representation, a user may view a projected 3D volume FOV and isocenter on acquired 2D scout images prior to the 3D image being acquired. For example, referring to FIGS. 2C through 2E, via the multiple FOV representation, the system 100 may display projected image volume 224 and isocenter on an extended localization image 201, such that a user may view the displayed information to acquisition of image volume 225. In another example, referring to FIGS. 3C through 3E, via the multiple FOV representation, the system 100 may display projected image volume 324 and isocenter on an extended localization image 301, such that a user may view the displayed information prior to acquisition of image volume 325.

Via the multiple FOV representation, the system 100 may display, to a user, how moving the imaging device 112 relates to the patient anatomy captured in a localization image (e.g., an extended localization image 201, one or more localization images 222, an extended localization image 301, one or more localization images 322, etc.). Adjusting the O-arm position while using the FOV preview supported by aspects of the present disclosure allows the user to see the change in isocenter without acquiring additional localization images.

In some aspects, the system 100 may support a user input (received at 531) with respect to the multiple FOV representation via the user interface 110 of the system 100. In some examples, the user input may include target coordinates, a target object, target boundaries, and the like. In some examples, the target coordinates and/or target boundaries may correspond to a region of interest (e.g., regions A or B described with reference to FIGS. 2A through 2G, region C described with reference to FIGS. 3A through 3E, etc.).

At 535, the system 100 may generate movement data associated with positioning or orienting components (e.g., source 138, detector 140, rotor 142, collimator 144, etc.) of the imaging device 112 in association with capturing the image volume. In some aspects, the system 100 may generate the movement data based on the target boundaries, target coordinates corresponding to the target boundaries, spatial coordinates associated with the image volume, spatial coordinates associated with the projected image volume, or a combination thereof.

For example, the system 100 may translate the user input (e.g., target boundaries, target coordinates, etc.) with respect to the multiple FOV representation into system positioning commands and source-detector acquisition motion. In some aspects, the user input may include an indication of a target center for the image volume, and the system 100 may determine the translation of the source 138 and detector 140 in association with capturing the image volume. For example, the system 100 may generate the movement data for positioning or orienting the imaging device 112 to capture the image volume with respect to the target center.

In some aspects, the system 100 may generate the movement data based on the target boundaries, image orientation information (e.g., orientation information associated with the fused localization image), and a system geometry associated with the imaging device 112 (or a system geometry of an imaging system including the imaging device 112). It is to be understood that references to the imaging device 112 may be applied to an imaging system associated with the imaging device 112.

In an example of generating the movement data, the system 100 may combine the target boundaries defined on the fused localization image with the image orientation information and known system geometry. The system 100 may generate the movement data based on the combination of the target boundaries, the image orientation information, and the known system geometry. In some aspects, the image orientation information may include patient orientation relative to imaging device 112 (e.g., left/right, prone/supine, etc.), plane of focus for the patient anatomy in a 2D image, and image pixel size. In some examples, with reference to the plane of focus for the 2D image, the system 100 may assume isocenter for the plane of focus. In some cases, assuming the isocenter for the plane of focus may impact motion accuracy associated with generating movement data for positioning or orienting the imaging device 112.

In an example of generating the movement data, the system 100 may determine system axes of motion based on image view associated with the fused localization image. For example, for an anterior-posterior (AP) view associated with a patient 148, the system 100 may determine movement data with respect to X and Y axes of the coordinate system 101. In another example, for a lateral (LAT) view associated with the patient 148, the system 100 may determine movement data with respect to Z and X axes of the coordinate system 101. It is to be understood that the views (e.g., AP view, LAT view) axes described with reference to the movement data are non-limiting examples, and aspects of the present disclosure are not limited thereto.

At 540, the system 100 may generate settings associated with capturing the image volume. In some aspects, the system 100 may generate the settings based on the target boundaries, the target coordinates, and image data of the fused localization image. For example, the system 100 may apply the settings to the imaging device 112. In an example, the system 100 may translate a user selected region associated with the fused localization image to image settings for acquiring an image volume (e.g., image volume 225, image volume 325, etc.) as described herein.

According to example aspects of the present disclosure, the system 100 may support manual and automatic movement of the imaging device 112 in association with capturing an image volume (e.g., image volume 225, image volume 325, etc.) as described herein.

For example, at 545, the system 100 may provide guidance information associated with capturing the image volume. In an example, the guidance information may include the movement data (as generated at 535), the spatial coordinates, or both. In some aspects, the system 100 may provide any combination of visual, audible, and haptic prompts (e.g., via user interface 110) for providing the guidance information to a user.

In an example implementation, at 547, a user may provide user controlled movement associated with positioning one or more components of the imaging device 112.

In another example, at 550, the system 100 may control movement of one or more components (e.g., source 138, detector 140, rotor 142, collimator 144, mobile cart 153, etc.) of the imaging device 112 based on the movement data. That is, for example, the system 100 may automatically control the movement based on the movement data.

At 555, the system 100 may capture an image volume (e.g., image volume 225, image volume 325) including based on the target boundaries associated with the fused localization image. For example, with reference to FIGS. 2A through 2G, the image volume 225 may include object 215 (including regions A and B). In another example, with reference to FIGS. 3A through 3E, the image volume 325 may include object 315 (including region C). As described herein, the image volume may be based on three-dimensional system coordinates of the imaging device 112.

In some aspects, the patient 148 is positioned along the geometric isocenter 203 of the imaging system. In some aspects, with reference to the examples associated with FIGS. 2A through 2G, a virtual isocenter associated with capturing an image volume as described herein (e.g., image volume 225) may be the same as the geometric isocenter 203. In some other aspects, with reference to the examples associated with FIGS. 3A through 3E, a virtual isocenter associated with capturing an image volume as described herein (e.g., image volume 325) may be different from the geometric isocenter 203.

In some aspects, capturing the image volume (at 555) may include compensating for parallax associated with the different points of view associated with capturing localization images. For example, with reference to the examples associated with FIGS. 2A through 2G, the imaging device 112 may capture localization images 222 according to different respective points of view from the source 138. In an example, for cases in which the imaging device 112 captures the localization images 222 without using a slot filter, parallax may result when merging (stitching) the different localization images 222.

In an example, the system 100 may stitch the localization images 222 by projecting the peripheral acquisitions onto a plane, rather than an arc along which projections are acquired. Through the projection of the peripheral acquisitions onto the plane, the system 100 may account for magnification effects that would otherwise introduce distortion in the stitching of the localization images 222. Aspects of the present disclosure support similarly applying the features of parallax compensation to the examples associated with FIGS. 3A through 3E.

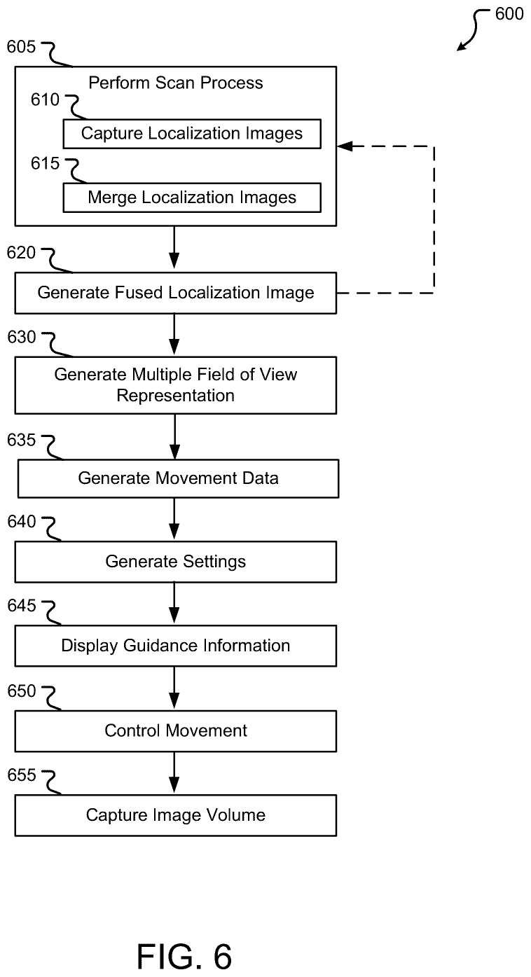

FIG. 6 illustrates an example of a process flow 600 in accordance with aspects of the present disclosure.

In some examples, process flow 600 may be implemented by aspects of the system 100 described herein.

In the following description of the process flow 600, the operations may be performed in a different order than the order shown, the operations may be performed in different orders or at different times, or one or more operations may be repeated. Certain operations may also be left out of the process flow 600, or other operations may be added to the process flow 600.

It is to be understood that any device (e.g., computing device 102, imaging device 112, etc.) of the system 100 may perform the operations shown.

Aspects of the process flow 600 may be implemented by an imaging system including: a processor coupled with the imaging system; and memory coupled with the processor and storing data thereon that, when executed by the processor, enable the processor to perform operations of the process flow 600.

At 605, the process flow 600 may include performing a scan process of a patient anatomy according to an extended width mode of capture.

In some aspects, performing the scan process according to the extended width mode of capture may include capturing (at 610) a set of localization images associated with the patient anatomy in association with the extended width mode of capture. In some other aspects, performing the scan process according to the extended width mode of capture may include merging (at 615) the set of localization images.

At 620, the process flow 600 may include generating a fused localization image of the patient anatomy based on performing the scan process.

In some aspects, the fused localization image is generated based on merging the set of localization images. In some aspects, the fused localization image includes a two-dimensional image. In some aspects, a width of the fused localization image is equal to or less than 80 centimeters.

In some aspects, the process flow 600 may include establishing one or more boundaries associated with the fused localization image based on: a first boundary associated with a first localization image of the set of localization images; and a second boundary associated with a second localization image of the set of localization images.

At 630, the process flow 600 may include generating a multiple FOV representation of the patient anatomy based on one or more target boundaries associated with the fused localization image, image data included in the fused localization image, and the image volume. In some aspects, the multiple FOV representation includes a preview representation of an image volume (also referred to herein as a projection of the image volume or projected image volume) including at least a portion of the patient anatomy.

In some example implementations, the process flow 600 may include repeating the features described at 605 and 620 for multiple projections (e.g., lateral, anterior-posterior, etc.). For example, the process flow 600 may include feeding the multiple projections into the multiple field of view representation of 630 to show multiple fields of view. In an example, for a first pass including 605 and 620, the process flow 600 may include generating a fused localization image of a first type (e.g., lateral view). In another example, for a second pass including 605 and 620, the process flow 600 may include generating a fused localization image of a second type (e.g., anterior-posterior view). At 630, the process flow 600 may include displaying the fused localization image of the first type (e.g., lateral view) and the fused localization image of the second type (e.g., anterior-posterior view) via the multiple field of view representation of 630.

At 635, the process flow 600 may include generating movement data associated with positioning or orienting at least one of a radiation source, a detector, and a rotor of the imaging system in association with capturing the image volume including at least a portion of the patient anatomy. In some aspects, generating the movement data is based on the one or more target boundaries associated with the fused localization image, target coordinates associated with the fused localization image, spatial coordinates associated with the image volume, or a combination thereof.

In some aspects, generating the movement data is based on: the one or more target boundaries associated with the fused localization image; orientation information associated with the fused localization image; and a system geometry associated with the imaging system.

At 640, the process flow 600 may include generating one or more settings associated with capturing the image volume based on: the one or more target boundaries associated with the fused localization image; the target coordinates associated with the fused localization image; and image data associated with the fused localization image.

At 645, the process flow 600 may include displaying guidance information associated with capturing the image volume at a user interface associated with the imaging system, wherein the guidance information includes at least one of the movement data and the spatial coordinates.

At 650, the process flow 600 may include controlling movement of at least one of the source, the detector, and the rotor based on the movement data (e.g., the system 100 may automatically control the movement). In some cases, the process flow 600 may include controlling the movement in response to a user input.

At 655, the process flow 600 may include capturing an image volume including at least a portion of the patient anatomy based on one or more target boundaries associated with the fused localization image.

In some aspects, the portion of the patient anatomy is included in one or more localization images of the set of localization images. In some aspects, the image volume is based on three-dimensional system coordinates of the imaging system.

In some aspects, a subject including the patient anatomy is positioned along an isocenter of the imaging system. In some aspects, a virtual isocenter associated with capturing the image volume is different from the isocenter of the imaging system.

In some aspects, capturing the image volume is in response to at least one of: a user input associated with the fused localization image; and a second user input associated with the multiple FOV representation of the patient anatomy, wherein the multiple FOV representation includes a preview representation of the image volume.

In some aspects, capturing the image volume (at 655) and/or generating the fused localization image (at 620) may include compensating for parallax associated with: a point of view associated with capturing a first localization image of the set of localization images; and a second point of view associated with capturing a second localization image of the set of localization images.

The process flow 600 (and/or one or more operations thereof) may be carried out or otherwise performed, for example, by at least one processor. The at least one processor may be the same as or similar to the processor(s) 104 of the computing device 102 described above. The at least one processor may be part of an imaging system (including the imaging device 112), a robot (such as a robot 114) or part of a navigation system (such as a navigation system 118). A processor other than any processor described herein may also be used to execute the process flow 600. The at least one processor may perform operations of the process flow 600 by executing elements stored in a memory such as the memory 106. The elements stored in memory and executed by the processor may cause the processor to execute one or more operations of a function as shown in the process flow 600. One or more portions of the process flow 600 may be performed by the processor executing any of the contents of memory, such as image processing 120, a segmentation 122, a transformation 124, a registration 128, and/or object detection 129.

As noted above, the present disclosure encompasses methods with fewer than all of the features identified in FIGS. 5 and 6 (and the corresponding description of the process flows 500 and 600), as well as methods that include additional features beyond those identified in FIGS. 5 and 6 (and the corresponding description of the process flows 500 and 600). The present disclosure also encompasses methods that include one or more steps from one method described herein, and one or more steps from another method described herein. Any correlation described herein may be or include a registration or any other correlation.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example aspects of the present disclosure include:

An imaging system including: a processor coupled with the imaging system; and memory coupled with the processor and storing data thereon that, when executed by the processor, enable the processor to: perform a scan process of a patient anatomy according to an extended width mode of capture; generate a fused localization image of the patient anatomy based on performing the scan process; and capture an image volume including at least a portion of the patient anatomy based on one or more target boundaries associated with the fused localization image.

Any of the aspects herein, wherein the data executable to perform the scan process according to the extended width mode of capture is further executable to: capture a set of localization images associated with the patient anatomy in association with the extended width mode of capture; and merge the set of localization images, wherein: the fused localization image is generated based on merging the set of localization images; and the portion of the patient anatomy is included in one or more localization images of the set of localization images.

Any of the aspects herein, wherein the data executable to capture the image volume is further executable to compensate for parallax associated with: a point of view associated with capturing a first localization image of the set of localization images; and a second point of view associated with capturing a second localization image of the set of localization images.

Any of the aspects herein, wherein the data is further executable by the processor to establish one or more boundaries associated with the fused localization image based on: a first boundary associated with a first localization image of the set of localization images; and a second boundary associated with a second localization image of the set of localization images.

Any of the aspects herein, wherein the data is further executable by the processor to: generate movement data associated with positioning or orienting at least one of a radiation source, a detector, and a rotor of the imaging system in association with capturing the image volume, wherein generating the movement data is based on the one or more target boundaries, target coordinates associated with the fused localization image, spatial coordinates associated with the image volume, or a combination thereof.

Any of the aspects herein, wherein the data is further executable by the processor to: display guidance information associated with capturing the image volume at a user interface associated with the imaging system, wherein the guidance information includes at least one of the movement data and the spatial coordinates.

Any of the aspects herein, wherein the data is further executable by the processor to: control movement of at least one of the source, the detector, and the rotor based on the movement data.

Any of the aspects herein, wherein generating the movement data is based on: the one or more target boundaries associated with the fused localization image; orientation information associated with the fused localization image; and a system geometry associated with the imaging system.

Any of the aspects herein, wherein the data is further executable by the processor to generate one or more settings associated with capturing the image volume based on: the one or more target boundaries associated with the fused localization image; target coordinates associated with the fused localization image; and image data associated with the fused localization image.

Any of the aspects herein, wherein: a subject including the patient anatomy is positioned along an isocenter of the imaging system; and a virtual isocenter associated with capturing the image volume is different from the isocenter of the imaging system.

Any of the aspects herein, wherein the data is further executable by the processor to: generate a multiple field of view representation of the patient anatomy based on the one or more target boundaries associated with the fused localization image, image data included in the fused localization image, and the image volume, wherein the multiple field of view representation includes a preview representation of the image volume.

Any of the aspects herein, wherein capturing the image volume is in response to at least one of: a user input associated with the fused localization image; and a second user input associated with a multiple field of view representation of the patient anatomy, wherein the multiple field of view representation includes a preview representation of the image volume.

Any of the aspects herein, wherein the fused localization image includes a two-dimensional image.

Any of the aspects herein, wherein the image volume is based on three-dimensional system coordinates of the imaging system.

Any of the aspects herein, wherein a width of the fused localization image is equal to or less than 80 centimeters.

A system including: an imaging device; a processor; and memory coupled with the processor and storing data thereon that, when executed by the processor, enable the processor to: perform a scan process of a patient anatomy according to an extended width mode of capture; generate a fused localization image of the patient anatomy based on performing the scan process; and capture an image volume including at least a portion of the patient anatomy based on one or more target boundaries associated with the fused localization image.

Any of the aspects herein, wherein the data executable to perform the scan process according to the extended width mode of capture is further executable to: capture a set of localization images associated with the patient anatomy in association with the extended width mode of capture; and merge the set of localization images, wherein: the fused localization image is generated based on merging the set of localization images; and the portion of the patient anatomy is included in one or more localization images of the set of localization images.

Any of the aspects herein, wherein the data is further executable by the processor to: generate movement data associated with positioning or orienting the imaging device in association with capturing the image volume, wherein generating the movement data is based on the one or more target boundaries, target coordinates associated with the fused localization image, spatial coordinates associated with the image volume, or a combination thereof.

A method including: performing, at an imaging system, a scan process of a patient anatomy according to an extended width mode of capture; generating, at the imaging system, a fused localization image of the patient anatomy based on performing the scan process; and capturing, at the imaging system, an image volume including at least a portion of the patient anatomy based on one or more target boundaries associated with the fused localization image.

Any of the aspects herein, wherein performing the scan process according to the extended width mode of capture includes: capturing a set of localization images associated with the patient anatomy in association with the extended width mode of capture; and merging the set of localization images, wherein: the fused localization image is based on merging the set of localization images; and the portion of the patient anatomy is included in one or more localization images of the set of localization images.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electromagnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. An imaging system comprising:
a processor coupled with the imaging system; and
memory coupled with the processor and storing data thereon that, when executed by the processor, enable the processor to:
perform a scan process of a patient anatomy according to an extended width mode of capture;
generate a fused localization image of the patient anatomy based on performing the scan process; and
capture an image volume comprising at least a portion of the patient anatomy based on one or more target boundaries associated with the fused localization image.

2. The imaging system of claim 1, wherein the data executable to perform the scan process according to the extended width mode of capture is further executable to:
capture a set of localization images associated with the patient anatomy in association with the extended width mode of capture; and
merge the set of localization images,
wherein:
the fused localization image is generated based on merging the set of localization images; and
the portion of the patient anatomy is comprised in one or more localization images of the set of localization images.

3. The imaging system of claim 2, wherein the data executable to capture the image volume is further executable to compensate for parallax associated with:
a point of view associated with capturing a first localization image of the set of localization images; and
a second point of view associated with capturing a second localization image of the set of localization images.

4. The imaging system of claim 2, wherein the data is further executable by the processor to establish one or more boundaries associated with the fused localization image based on:
a first boundary associated with a first localization image of the set of localization images; and
a second boundary associated with a second localization image of the set of localization images.

5. The imaging system of claim 1, wherein the data is further executable by the processor to:
generate movement data associated with positioning or orienting at least one of a radiation source, a detector, and a rotor of the imaging system in association with capturing the image volume, wherein generating the movement data is based on the one or more target boundaries, target coordinates associated with the fused localization image, spatial coordinates associated with the image volume, or a combination thereof.

6. The imaging system of claim 5, wherein the data is further executable by the processor to:
display guidance information associated with capturing the image volume at a user interface associated with the imaging system, wherein the guidance information comprises at least one of the movement data and the spatial coordinates.

7. The imaging system of claim 5, wherein the data is further executable by the processor to:
control movement of at least one of the radiation source, the detector, and the rotor based on the movement data.

8. The imaging system of claim 5, wherein generating the movement data is based on:
the one or more target boundaries associated with the fused localization image;
orientation information associated with the fused localization image; and
a system geometry associated with the imaging system.

9. The imaging system of claim 1, wherein the data is further executable by the processor to generate one or more settings associated with capturing the image volume based on:
the one or more target boundaries associated with the fused localization image;
target coordinates associated with the fused localization image; and
image data associated with the fused localization image.

10. The imaging system of claim 1, wherein:
a subject comprising the patient anatomy is positioned along an isocenter of the imaging system; and
a virtual isocenter associated with capturing the image volume is different from the isocenter of the imaging system.

11. The imaging system of claim 1, wherein the data is further executable by the processor to:
generate a multiple field of view representation of the patient anatomy based on the one or more target boundaries associated with the fused localization image, image data comprised in the fused localization image, and the image volume,
wherein the multiple field of view representation comprises a preview representation of the image volume.

12. The imaging system of claim 1, wherein capturing the image volume is in response to at least one of:
a user input associated with the fused localization image; and
a second user input associated with a multiple field of view representation of the patient anatomy, wherein the multiple field of view representation comprises a preview representation of the image volume.

13. The imaging system of claim 1, wherein the fused localization image comprises a two-dimensional image.

14. The imaging system of claim 1, wherein the image volume is based on three-dimensional system coordinates of the imaging system.

15. The imaging system of claim 1, wherein a width of the fused localization image is equal to or less than 80 centimeters.

16. A system comprising:
an imaging device;
a processor; and
memory coupled with the processor and storing data thereon that, when executed by the processor, enable the processor to:
perform a scan process of a patient anatomy according to an extended width mode of capture;
generate a fused localization image of the patient anatomy based on performing the scan process; and
capture an image volume comprising at least a portion of the patient anatomy based on one or more target boundaries associated with the fused localization image.

17. The system of claim 16, wherein the data executable to perform the scan process according to the extended width mode of capture is further executable to:
capture a set of localization images associated with the patient anatomy in association with the extended width mode of capture; and
merge the set of localization images, wherein:
the fused localization image is generated based on merging the set of localization images; and
the portion of the patient anatomy is comprised in one or more localization images of the set of localization images.

18. The system of claim 16, wherein the data is further executable by the processor to:
generate movement data associated with positioning or orienting the imaging device in association with capturing the image volume, wherein generating the movement data is based on the one or more target boundaries, target coordinates associated with the fused localization image, spatial coordinates associated with the image volume, or a combination thereof.

19. A method comprising:
performing, at an imaging system, a scan process of a patient anatomy according to an extended width mode of capture;
generating, at the imaging system, a fused localization image of the patient anatomy based on performing the scan process; and
capturing, at the imaging system, an image volume comprising at least a portion of the patient anatomy based on one or more target boundaries associated with the fused localization image.

20. The method of claim 19, wherein performing the scan process according to the extended width mode of capture comprises:
capturing a set of localization images associated with the patient anatomy in association with the extended width mode of capture; and
merging the set of localization images, wherein:
the fused localization image is based on merging the set of localization images; and
the portion of the patient anatomy is comprised in one or more localization images of the set of localization images.
